(19) United States
(12) Patent Application Publication
(10) Pub. No.: US 2026/0333982 A1
(43) Pub. Date: Oct. 22, 2026

(54) Light generating device comprising LED package configurations for improved disinfection

(71) Applicant: SIGNIFY HOLDING B.V., EINDHOVEN (NL)

(72) Inventors: TIES VAN BOMMEL, HORST (NL); ERIK PETRUS JOHANNES MALLENS, DEN BOSCH (NL)

(21) Appl. No.: 19/486,089

(22) PCT Filed: Jun. 10, 2024
(86) PCT No.: PCT/EP2024/065961

§ 371 Date: Nov. 20, 2025

(30) Foreign Application Priority Data
Jun. 15, 2023 (EP) 23179499.1

(51) Int. Cl. H10H 29/851; A61L 2/084; A61L 2/085; A61L 2/101; H05B 45/20; H05B 47/16; H05B 47/19; H10H 20/851

(52) U.S. Cl. CPC H10H 29/8513; A61L 2/084; A61L 2/085; A61L 2/101; H10H 20/8511; A61L 2202/11; H05B 45/20; H05B 47/16; H05B 47/19

(57) ABSTRACT

The invention provides a light generating system (1000) configured to generate system light (1001), wherein the light generating system (1001) comprises a first light generating device (110) and a second light generating device (120); wherein: —the first light generating device (110) comprises a first light source (10) and a first luminescent converter (210); wherein the first luminescent converter (210) comprises a first matrix material (215) and a first luminescent material (216), wherein the first luminescent material (216) has a first weight percentage CW1 relative to a total weight of the first luminescent converter (216); the first light generating device (110) is configured to generate first device light (111) having a spectral power distribution in the wavelength range of 380-780 nm with at least 60% of a spectral power provided by the first light source light (11) and at maximum 40% of the spectral power provided by the first converter light (211); —the second light generating device (120) comprises a second light source (20) and a second luminescent converter (220); wherein the second luminescent converter (220) comprises a second matrix material (225) and a second luminescent material (226), wherein the second luminescent material (226) has a second weight percentage CW₂ relative to a total weight of the second luminescent converter (220); the second light generating device (120) is configured to generate second device light (121) having a spectral power distribution in the wavelength range of 380-780 nm with at least 60% of the spectral power provided by the second converter light (221) and at maximum 40% of the spectral power provided by the second light source light (21); and —CW₁/CW₂≤0.5.

### FIELD OF THE INVENTION

The invention relates to a light generating system and to a lighting device comprising such light generating system. Yet, the invention also relates to a method for treating at least part of a space or of an object.

### BACKGROUND OF THE INVENTION

Antibacterial light sources are known in the art. US2020390915, for instance, describes a light source for emitting emitted light having an SPD comprising: (a) a plurality of light emitters including at least one violet solid-state emitter; (b) at least one phosphor; wherein said light emitters and said at least one phosphor being configured such that: at least 25% of the power within the SPD is in the range 390-420 nm, and the emitted light has a chromaticity which is within a Duv distance of less than 5 points from the Planckian locus.

### SUMMARY OF THE INVENTION

UV light has been used for disinfection for over 100 years. Wavelengths between about 190 nm and 300 nm may be strongly absorbed by nucleic acids, which may result in defects in an organism's genome. This may be desired for inactivating (killing), bacteria and viruses, but may also have undesired side effects for humans. Therefore, the selection of wavelength of radiation, intensity of radiation and duration of irradiation may be limited in environments where people may reside such as offices, public transport, cinema's, restaurants, shops, etc., thus limiting the disinfection capacity. Especially in such environments, additional measures of disinfection may be advantageous to prevent the spread of bacteria and viruses such as influenza or novel (corona) viruses like COVID-19, SARS and MERS

It appears desirable to produce systems, which provide alternative ways for air treatment, such as disinfection. Further, existing systems for disinfection may not easily be implemented in existing infrastructure, such as in existing buildings like offices, hospitality areas, etc. and/or may not easily be able to serve larger spaces. This may again increase the risk of contamination. Further, incorporation in HVAC systems may not lead to desirable effects and appears to be relatively complex. Further, existing systems may not be efficient, or may be relatively bulky, and may also not easily be incorporated in functional devices, such as e.g. luminaires. Other disinfection systems may use one or more anti-microbial and/or anti-viral means to disinfect a space or an object. Examples of such means may be chemical agents which may raise concerns. For instance, the chemical agents may also be harmful for people and pets.

In embodiments, the disinfecting light, may especially comprise ultraviolet (UV) radiation (and/or optionally violet radiation), i.e., the light may comprise a wavelength selected from the ultraviolet wavelength range (and/or optionally the violet wavelength range). However, other wavelengths are herein not excluded. The ultraviolet wavelength range is defined as light in a wavelength range from 100 to 380 nm and can be divided into different types of UV light/UV wavelength ranges (Table 1). Different UV wavelengths of radiation may have different properties and thus may have different compatibility with human presence and may have different effects when used for disinfection (Table 1).

TABLE 1
Properties of different types of UV, violet, and NIR wavelength light

| | | | (Relative) | | | | |
|---|---|---|---|---|---|---|---|
| | | | sterilization | | | | |
| | Short | Wavelength | effectiveness | | Safe | Vitamin D | Ozone |
| Name | name | (nm) | Bacteria | Viruses | Radiation | generation | generation |
| Near | NIR | 780-950 | + | +/− | +++ | | |
| Infrared | | | | | | | |
| (deep red) | | | | | | | |
| Violet | V | 380-420 | +/− | − | + | | |
| Ultra- | UV-A | 315-380 | + | − | + | | |
| violet A | | | | | | | |
| Ultra- | UV-B | 280-315 | + | +/− | +/− | + | |
| violet B | | | | | | | |
| Near | Near | 230-280 | ++ | ++ | − | | |
| ultra- | UV-C | | | | | | |
| violet C | | | | | | | |
| Far | Far | 190-230 | +++ | +++ | + | | +/− |
| ultra- | UV-C | | | | | | |
| violet C | | | | | | | |
| Extreme | Extreme | 100-190 | +++ | +++ | − | | + |
| ultra- | UV-C | | | | | | |
| violet C | | | | | | | |

Each UV type/wavelength range may have different benefits and/or drawbacks. Relevant aspects may be (relative) sterilization effectiveness, safety (regarding radiation), and ozone production (as result of its radiation). Depending on an application a specific type of UV light or a specific combination of UV light types may be selected and provides superior performance over other types of UV light. UV-A may be (relatively) safe and may inactivate (kill) bacteria, but may be less effective in inactivating (killing) viruses. UV-B may be (relatively) safe when a low dose (i.e. low exposure time and/or low intensity) is used, may inactivate (kill) bacteria, and may be moderately effective in inactivating (killing) viruses. UV-B may also have the additional benefit that it can be used effectively in the production of vitamin D in a skin of a person or animal. Near UV-C may be relatively unsafe, but may effectively inactivating, especially kill bacteria and viruses. Far UV-C may also be effective in inactivating (killing) bacteria and viruses, but may be (relatively to other UV-C wavelength ranges) (rather) safe. Far-UV light may generate some ozone which may be harmful for human beings and animals. Extreme UV-C may also be effective in inactivating (killing) bacteria and viruses, but may be relatively unsafe. Extreme UV-C may generate ozone which may be undesired when exposed to human beings or animals. In some application ozone may be desired and may contribute to disinfection, but then its shielding from humans and animals may be desired. Hence, in the table “+” for ozone production especially implies that ozone is produced which may be useful for disinfection applications, but may be harmful for humans/animals when they are exposed to it. Hence, in many applications this “+” may actually be undesired while in others, it may be desired. The types of light indicated in above table may in embodiments be used to sanitize air and/or surfaces.

The terms “inactivating” and “killing” with respect to a virus may herein especially refer to damaging the virus in such a way that the virus can no longer infect and/or reproduce in a host cell, i.e., the virus may be (essentially) harmless after inactivation or killing.

Hence, in embodiments, the light may comprise a wavelength in the UV-A range. In further embodiments, the light may comprise a wavelength in the UV-B range. In further embodiments, the light may comprise a wavelength in the Near UV-C range. In further embodiments, the light may comprise a wavelength in the Far UV-C range. In further embodiments, the light may comprise a wavelength in the extreme UV-C range. The Near UV-C, the Far UV-C and the extreme UV-C ranges may herein also collectively be referred to as the UV-C range. Hence, in embodiments, the light may comprise a wavelength in the UV-C range. In other embodiments, the light may comprise violet radiation.

Hence, light or radiation described herein may also be indicated as disinfection light (unless otherwise indicated or clear from the context).

Using e.g. violet light may lead to a source of light that has no substantial lighting function, let alone a general lighting function. Hence, one could imagine integrating such light source in a light generating device. However, this may have undesirable impact on the spectral properties of the light. Further, it may be desirable to apply the violet light at some locations and/or during specific time periods, or as function of a sensor signal, and not to apply it when it is not necessary. Current lighting systems may not provide one or more of these function. Hence, it is an aspect of the invention to provide an alternative light generating system, which preferably further at least partly obviates one or more of above-described drawbacks. The present invention may have as object to overcome or ameliorate at least one of the disadvantages of the prior art, or to provide a useful alternative.

According to a first aspect, the invention provides a light generating system (“system”) configured to generate system light, wherein the light generating system comprises a first light generating device and a second light generating device. In embodiments, the first light generating device may comprises a first light source and a first luminescent converter. Especially, in embodiments the first light source may comprise a solid state light source. Further, the first light source may be configured to generate first light source light having a first light source centroid wavelength (λ_{c,S1}) especially selected from the wavelength range of 380-420 nm. Yet, in embodiments the first luminescent converter may be configured to convert at least part of the first light source light into first luminescent converter light having a first luminescent converter centroid wavelength (λ_{c,L1}) especially selected from the wavelength range of 590-780 nm. Yet, in embodiments the first luminescent converter may comprise a first matrix material and a first luminescent material. Especially, the first luminescent material may have a first weight percentage CW₁ relative to a total weight of the first luminescent converter. In specific embodiments, the first light generating device may be configured to generate first device light having a spectral power distribution in the wavelength range of 380-780 nm with, in embodiments at least 60% of a spectral power provided by the first light source light and at maximum 40% of the spectral power provided by the first converter light. In embodiments, the second light generating device may comprise a second light source and a second luminescent converter. Especially, in embodiments the second light source may comprise a solid state light source. Further, the second light source may be configured to generate second light source light having a second light source centroid wavelength (λ_{c,S2}) especially selected from the wavelength range of 430-490 nm. Yet, in embodiments the second luminescent converter may be configured to convert at least part of the second light source light into second converter light having a second luminescent converter centroid wavelength (λ_{c,L2}) especially selected from the wavelength range of 490-780 nm. Yet, in embodiments the second luminescent converter may comprise a second matrix material and a second luminescent material, wherein the second luminescent material may have a second weight percentage CW₂ relative to a total weight of the second luminescent converter. In specific embodiments, the second light generating device may be configured to generate second device light having a spectral power distribution in the wavelength range of 380-780 nm with, in embodiments at least 60% of the spectral power provided by the second converter light and at maximum 40% of the spectral power provided by the second light source light. Further, in specific embodiments CW₁/CW₂≤0.5 may apply. Therefore, in specific embodiments the invention provides a light generating system configured to generate system light, wherein the light generating system comprises a first light generating device and a second light generating device; wherein: (A) the first light generating device comprises a first light source and a first luminescent converter; wherein the first light source comprises a solid state light source, wherein the first light source is configured to generate first light source light having a first light source centroid wavelength (λ_{c,S1}) selected from the wavelength range of 380-420 nm; wherein the first luminescent converter is configured to convert at least part of the first light source light into first luminescent converter light having a first luminescent converter centroid wavelength (λ_{c,L1}) selected from the wavelength range of 590-780 nm; wherein the first luminescent converter comprises a first matrix material and a first luminescent material, wherein the first luminescent material has a first weight percentage CW₁ relative to a total weight of the first luminescent converter; (B) the first light generating device is configured to generate first device light having a spectral power distribution in the wavelength range of 380-780 nm with at least 60% of a spectral power provided by the first light source light and at maximum 40% of the spectral power provided by the first converter light; (C) the second light generating device comprises a second light source and a second luminescent converter; wherein the second light source comprises a solid state light source, wherein the second light source is configured to generate second light source light having a second light source centroid wavelength (λ_{c,S2}) selected from the wavelength range of 430-490 nm; wherein the second luminescent converter is configured to convert at least part of the second light source light into second converter light having a second luminescent converter centroid wavelength (λ_{c,L2}) selected from the wavelength range of 490-780 nm; wherein the second luminescent converter comprises a second matrix material and a second luminescent material, wherein the second luminescent material has a second weight percentage CW₂ relative to a total weight of the second luminescent converter; (D) the second light generating device is configured to generate second device light having a spectral power distribution in the wavelength range of 380-780 nm with at least 60% of the spectral power provided by the second converter light and at maximum 40% of the spectral power provided by the second light source light; and (E) CW₁/CW₂≤0.5.

With such system, white light may be provided having a disinfection function. Further, with such system, white light may be provided having a controllable color point and/or color temperature. Yet further, a color rendering index (CRI) of the system light may be relatively high, e.g. at least about 82, such as at least about 85. With such light generating system, white light can be generated in a relatively efficient way and also having a relatively high color gamut and/or improved whiteness perception. With the use of at least two light generating devices, wherein luminescent materials may be provided at different locations, cross-talk may be prevented. Further, the extraction efficiency may be increased. The first luminescent material may promote extraction of violet light from the first luminescent converter, e.g. due to a difference in refractive index and/or due to reflection at luminescent material particles (embedded in the matrix). Such as system may provide improved disinfection e.g. due to increased extraction efficiency because of the low concentration of the first luminescent material in the first luminescent converter.

As indicated above, the light generating system may especially comprise a first light generating device and a second light generating device. Each light generating device may comprise a (solid state) light source. Embodiments of light generating devices and (solid state) light sources are described below in general, and may (individually) apply to the first light generating device and/or the second light generating device.

The term “light source” may in principle relate to any light source known in the art. It may be a conventional (tungsten) light bulb, a low pressure mercury lamp, a high pressure mercury lamp, a fluorescent lamp, an LED (light emitting diode). In a specific embodiment, the light source comprises a solid state light source (such as an LED or laser diode (or “diode laser”)). The term “light source” may also relate to a plurality of light sources, such as 2-2000 (solid state) LED light sources. Hence, the term LED may also refer to a plurality of LEDs. Further, the term “light source” may in embodiments also refer to a so-called chip-on-board (COB) light source. The term “COB” especially refers to LED chips in the form of a semiconductor chip that is neither encased nor connected but directly mounted onto a substrate, such as a PCB. Hence, a plurality of light emitting semiconductor light source may be configured on the same substrate. In embodiments, a COB is a multi LED chip configured together as a single lighting module. The term “light source” may also refer to a chip scaled package (CSP). A CSP may comprise a single solid state die with provided thereon a luminescent material comprising layer. The term “light source” may also refer to a midpower package. A midpower package may comprise one or more solid state die(s). The die(s) may be covered by a luminescent material comprising layer. The die dimensions may be equal to or smaller than 2 mm, such as in the range of e.g. 0.2-2 mm. Hence, in embodiments the light source comprises a solid state light source. Further, in specific embodiments, the light source comprises a chip scale packaged LED. Herein, the term “light source” may also especially refer to a small solid state light source, such as having a mini size or micro size. For instance, the light sources may comprise one or more of mini LEDs and micro LEDs. Especially, in embodiment the light sources comprise micro LEDs or “microLEDs” or “μLEDs”. Herein, the term mini size or mini LED especially indicates to solid state light sources having dimensions, such as die dimension, especially length and width, selected from the range of 100 μm-1 mm. Herein, the term u size or micro LED especially indicates to solid state light sources having dimensions, such as die dimension, especially length and width, selected from the range of 100 μm and smaller.

The light source may have a light escape surface. Referring to conventional light sources such as light bulbs or fluorescent lamps, it may be an outer surface of a glass or a quartz envelope. For LED's it may for instance be the LED die, or when a resin is applied to the LED die, the outer surface of the resin. In principle, it may also be the terminal end of a fiber. The term escape surface especially relates to that part of the light source, where the light actually leaves or escapes from the light source. The light source is configured to provide a beam of light. This beam of light (thus) escapes from the light exit surface of the light source.

Likewise, a light generating device may comprise a light escape surface, such as an end window. Further, likewise a light generating system may comprise a light escape surface, such as an end window.

The term “light source” may refer to a semiconductor light-emitting device, such as a light emitting diode (LEDs), a resonant cavity light emitting diode (RCLED), a vertical cavity laser diode (VCSELs), an edge emitting laser, etc. . . . . The term “light source” may also refer to an organic light-emitting diode (OLED), such as a passive-matrix (PMOLED) or an active-matrix (AMOLED). In a specific embodiment, the light source comprises a solid-state light source (such as an LED or laser diode). In an embodiment, the light source comprises an LED (light emitting diode). The terms “light source” or “solid state light source” may also refer to a superluminescent diode (SLED). The term LED may also refer to a plurality of LEDs. The term “light source” may also relate to a plurality of (essentially identical (or different)) light sources, such as 2-2000 solid state light sources. In embodiments, the light source may comprise one or more micro-optical elements (array of micro lenses) downstream of a single solid-state light source, such as an LED, or downstream of a plurality of solid-state light sources (i.e. e.g. shared by multiple LEDs). In embodiments, the light source may comprise an LED with on-chip optics. In embodiments, the light source comprises pixelated single LEDs (with or without optics) (offering in embodiments on-chip beam steering).

In embodiments, the light source may be configured to provide primary radiation, which is used as such, such as e.g. a blue light source, like a blue LED, or a green light source, such as a green LED, and a red light source, such as a red LED. Such LEDs, which may not comprise a luminescent material (“phosphor”) may be indicated as direct color LEDs. In other embodiments, however, the light source may be configured to provide primary radiation and part of the primary radiation is converted into secondary radiation. Secondary radiation may be based on conversion by a luminescent material. The secondary radiation may therefore also be indicated as luminescent material radiation. The luminescent material may in embodiments be comprised by the light source, such as an LED with a luminescent material layer or dome comprising luminescent material. Such LEDs may be indicated as phosphor converted LEDs or PC LEDs (phosphor converted LEDs). In other embodiments, the luminescent material may be configured at some distance (“remote”) from the light source, such as an LED with a luminescent material layer not in physical contact with a die of the LED. Hence, in specific embodiments the light source may be a light source that during operation emits at least light at wavelength selected from the range of 380-470 nm. However, other wavelengths may also be possible. This light may partially be converted by the luminescent material.

In embodiments, the light generating device may comprise a luminescent material. In embodiments, the light generating device may comprise a PC LED. In other embodiments, the light generating device may comprise a direct LED (i.e. no phosphor). In embodiments, the light generating device may comprise a laser device, like a laser diode. In embodiments, the light generating device may comprise a superluminescent diode. Hence, in specific embodiments, the light source may be selected from the group of laser diodes and superluminescent diodes. In other embodiments, the light source may comprise an LED.

The light source may especially be configured to generate light source light having an optical axis (O), (a beam shape,) and a spectral power distribution. The light source light may in embodiments comprise one or more bands, having band widths as known for lasers.

The term “light source” may (thus) refer to a light generating element as such, like e.g. a solid state light source, or e.g. to a package of the light generating element, such as a solid state light source, and one or more of a luminescent material comprising element and (other) optics, like a lens, a collimator. A light converter element (“converter element” or “converter”) may comprise a luminescent material comprising element. For instance, a solid state light source as such, like a blue LED, is a light source. A combination of a solid state light source (as light generating element) and a light converter element, such as a blue LED and a light converter element, optically coupled to the solid state light source, may also be a light source (but may also be indicated as light generating device). Hence, a white LED is a light source (but may e.g. also be indicated as (white) light generating device).

The term “light source” herein may also refer to a light source comprising a solid state light source, such as an LED or a laser diode or a superluminescent diode. The term “light source” may (thus) in embodiments also refer to a light source that is (also) based on conversion of light, such as a light source in combination with a luminescent converter material. Hence, the term “light source” may also refer to a combination of an LED with a luminescent material configured to convert at least part of the LED radiation, or to a combination of a (diode) laser with a luminescent material configured to convert at least part of the (diode) laser radiation. In embodiments, the term “light source” may also refer to a combination of a light source, like an LED, and an optical filter, which may change the spectral power distribution of the light generated by the light source. Especially, the term “light generating device” may be used to address a light source and further (optical components), like an optical filter and/or a beam shaping element, etc.

The phrases “different light sources” or “a plurality of different light sources”, and similar phrases, may in embodiments refer to a plurality of solid-state light sources selected from at least two different bins. Likewise, the phrases “identical light sources” or “a plurality of same light sources”, and similar phrases, may in embodiments refer to a plurality of solid-state light sources selected from the same bin.

The term “solid state light source”, or “solid state material light source”, and similar terms, may especially refer to semiconductor light sources, such as a light emitting diode (LED), a laser diode, or a superluminescent diode. Especially, in embodiments the first light source and the second light source may be selected from the group of light emitting diodes, laser diodes, and superluminescent diodes.

The first light generating device may comprise a laser bank comprising a plurality of first laser diodes. The second light generating device may comprise a laser bank comprising a plurality of second laser diodes. Hence, in embodiments laser banks may be applied. Laser banks may also be used to boast the input power. Therefore, in embodiments the system may comprise a plurality of light generating devices configured in a laser bank. A laser bank may comprise a light emitting arrangement comprising an (2D) array of a plurality of laser diodes arranged on a thermally conductive carrier and a (lens array having a) plurality of collimator lenses corresponding to the laser diodes such that each laser diode of the plurality of laser diodes comprises a collimator lens for collimating laser light emitted by the laser diode. The arrangement may comprise a package architecture or a canned architecture. In case of the package architecture a laser diode chip array is arranged on the thermally conductive carrier. A plurality of electrodes may be present for electrically connecting the plurality of laser diodes. A single laser bank may comprise both first light sources and second light sources. However, also a first laser bank comprising first light sources and a second laser bank comprising second light sources may be applied.

In embodiments, the first light generating device may comprise a package comprising one or more (first) solid state light sources. Alternatively or additionally, the second light generating device may comprise an LED package comprising one or more (second) solid state light sources. For instance, in embodiments the system may comprise an integrated light source package, wherein the integrated light source package comprises a common support member configured to support the first light source(s) and/or the second light source(s), wherein in specific embodiments the common support member may comprise a thermally conductive support. The thermally conductive support may comprise one or more of a heatsink, a heat spreader, and a vapor chamber. Further, in embodiments both the first light generating device and the second light generating device may be configured in a housing. In specific embodiments, first light generating device and the second light generating device may be configured in a single light reflective cup.

Hence, in embodiments the invention provides a light generating device comprising (KSiF) LED package configurations, amongst others for improved disinfection.

In embodiments, the light generating system may be provided as LED package.

In embodiments, the first light generating device and the second light generating device may share a same support, such as a PCB.

In embodiments, the first light generating device may comprise a first light source and a first luminescent converter. Further, in embodiments the second light generating device may comprise a second light source and a second luminescent converter. Here below, some embodiments in relation to luminescent materials are described in general. A luminescent converter may be configured to generate luminescent converter light upon irradiation with the first light source light or the second light source light, respectively. A luminescent converter may (thus) comprise a luminescent material.

The term “luminescent material” especially refers to a material that can convert first radiation, especially one or more of UV radiation and blue radiation, into second radiation. In general, the first radiation and second radiation have different spectral power distributions. Hence, instead of the term “luminescent material”, also the terms “luminescent converter” or “converter” may be applied. In general, the second radiation has a spectral power distribution at larger wavelengths than the first radiation, which is the case in the so-called down-conversion. In specific embodiments, however the second radiation has a spectral power distribution with intensity at smaller wavelengths than the first radiation, which is the case in the so-called up-conversion.

In embodiments, the “luminescent material” may especially refer to a material that can convert radiation into e.g. visible and/or infrared light. For instance, in embodiments the luminescent material may be able to convert one or more of UV radiation and blue radiation, into visible light. The luminescent material may in specific embodiments also convert radiation into infrared radiation (IR). Hence, upon excitation with radiation, the luminescent material emits radiation. In general, the luminescent material will be a down converter, i.e. radiation of a smaller wavelength is converted into radiation with a larger wavelength (λex<λem), though in specific embodiments the luminescent material may comprise up-converter luminescent material, i.e. radiation of a larger wavelength is converted into radiation with a smaller wavelength (λex>λem).

In embodiments, the term “luminescence” may refer to phosphorescence. In embodiments, the term “luminescence” may also refer to fluorescence. Instead of the term “luminescence”, also the term “emission” may be applied. Hence, the terms “first radiation” and “second radiation” may refer to excitation radiation and emission (radiation), respectively. Likewise, the term “luminescent material” may in embodiments refer to phosphorescence and/or fluorescence.

The term “luminescent material” may also refer to a plurality of different luminescent materials. Examples of possible luminescent materials are indicated below. Hence, the term “luminescent material” may in specific embodiments also refer to a luminescent material composition. Instead of the term “luminescent material” also the term “phosphor” may be applied. These terms are known to the person skilled in the art.

In embodiments, luminescent materials are selected from garnets and nitrides, especially doped with trivalent cerium or divalent europium, respectively. The term “nitride” may also refer to oxynitride or nitridosilicate, etc. Alternatively or additionally, the luminescent material(s) may be selected from silicates, especially doped with divalent europium.

In specific embodiments the luminescent material comprises a luminescent material of the type A3B5O12:Ce, wherein A in embodiments comprises one or more of Y, La, Gd, Tb and Lu, especially (at least) one or more of Y, Gd, Tb and Lu, and wherein B in embodiments comprises one or more of Al, Ga, In and Sc. Especially, A may comprise one or more of Y, Gd and Lu, such as especially one or more of Y and Lu. Especially, B may comprise one or more of Al and Ga, more especially at least Al, such as essentially entirely Al. Hence, especially suitable luminescent materials are cerium comprising garnet materials. Embodiments of garnets especially include A3B5O12 garnets, wherein A comprises at least yttrium or lutetium and wherein B comprises at least aluminum. Such garnets may be doped with cerium (Ce), with praseodymium (Pr) or a combination of cerium and praseodymium; especially however with Ce. Especially, B may comprise aluminum (Al); however, in addition to aluminum, B may also partly comprise gallium (Ga) and/or scandium (Sc) and/or indium (In), especially up to about 20% of B, more especially up to about 10% of B (i.e. the B ions essentially consist of 90 or more mole % of Al and 10 or less mole % of one or more of Ga, Sc and In); B may especially comprise up to about 10% gallium. In another variant, B and O may at least partly be replaced by Si and N. The element A may especially be selected from the group consisting of yttrium (Y), gadolinium (Gd), terbium (Tb) and lutetium (Lu). Further, Gd and/or Tb are especially only present up to an amount of about 20% of A. In a specific embodiment, the garnet luminescent material comprises (Y1−xLux)3B5O12:Ce, wherein x is equal to or larger than 0 and equal to or smaller than 1. The term “:Ce”, indicates that part of the metal ions (i.e. in the garnets: part of the “A” ions) in the luminescent material is replaced by Ce. For instance, in the case of (Y1−xLux)3Al5O12:Ce, part of Y and/or Lu is replaced by Ce. This is known to the person skilled in the art. Ce will replace A in general for not more than 10%; in general, the Ce concentration will be in the range of 0.1 to 4%, especially 0.1 to 2% (relative to A). Assuming 1% Ce and 10% Y, the full correct formula could be (Y0.1Lu0.89Ce0.01)3Al5O12. Ce in garnets is substantially or only in the trivalent state, as is known to the person skilled in the art.

In embodiments, the luminescent material (thus) comprises A3B5O12 wherein in specific embodiments at maximum 10% of B—O may be replaced by Si—N.

In specific embodiments the luminescent material comprises (Yₓ₁A′ₓ₂Ceₓ₃)₃(Al_{y1}B′_{y2})₅O₁₂, wherein x1+x2+x3=1, wherein x3>0, wherein 0<x2+x3≤0.2, wherein y1+y2=1, wherein especially 0≤y2≤0.2, wherein A′ comprises one or more elements selected from the group consisting of lanthanides, and wherein B′ comprises one or more elements selected from the group consisting of Ga, In and Sc. In embodiments, x3 is selected from the range of 0.001-0.1. In the present invention, especially x1>0, such as >0.2, like at least 0.8. Garnets with Y may provide suitable spectral power distributions.

In specific embodiments at maximum 10% of B—O may be replaced by Si—N. Here, B in B—O refers to one or more of Al, Ga, In and Sc (and O refers to oxygen); in specific embodiments B—O may refer to Al—O. As indicated above, in specific embodiments x3 may be selected from the range of 0.001-0.04. Especially, such luminescent materials may have a suitable spectral distribution (see however below), have a relatively high efficiency, have a relatively high thermal stability, and allow a high CRI (optionally in combination with (the) light of other sources of light as described herein). Hence, in specific embodiments A may be selected from the group consisting of Lu and Gd. Alternatively or additionally, B may comprise Ga. Hence, in embodiments the luminescent material comprises (Yₓ₁(Lu,Gd)ₓ₂Ceₓ₃)₃(Al_{y1}Ga_{y2})₅O₁₂, wherein Lu and/or Gd may be available. Even more especially, x3 is selected from the range of 0.001-0.1, wherein 0<x2+x3≤0.1, and wherein 0≤y2≤0.1. Further, in specific embodiments, at maximum 1% of B—O may be replaced by Si—N. Here, the percentage refers to moles (as known in the art); see e.g. also EP3149108. In yet further specific embodiments, the luminescent material comprises (Yₓ₁Ceₓ₃)₃Al₅O₁₂, wherein x1+x3=1, and wherein 0<x3≤0.2, such as 0.001-0.1.

In specific embodiments, the light generating device may only include luminescent materials selected from the type of cerium comprising garnets. In even further specific embodiments, the light generating device includes a single type of luminescent materials, such as (Yₓ₁A′ₓ₂Ceₓ₃)₃(Al_{y1}B′_{y2})₅O₁₂. Hence, in specific embodiments the light generating device comprises luminescent material, wherein at least 85 weight %, even more especially at least about 90 wt. %, such as yet even more especially at least about 95 weight % of the luminescent material comprises (Yₓ₁A′ₓ₂Ceₓ₃)₃ (Al_{y1}B′y2)₅O₁₂. Here, wherein A′ comprises one or more elements selected from the group consisting of lanthanides, and wherein B′ comprises one or more elements selected from the group consisting of Ga, In and Sc, wherein x1+x2+x3=1, wherein x3>0, wherein 0<x2+x3≤0.2, wherein y1+y2=1, wherein 0≤y2≤0.2. Especially, x3 is selected from the range of 0.001-0.1. Note that in embodiments x2=0. Alternatively or additionally, in embodiments y2=0.

In specific embodiments, A may especially comprise at least Y, and B may especially comprise at least Al.

Alternatively or additionally, the luminescent material may comprise a luminescent material of the type A₃Si₆N₁₁:Ce³⁺, wherein A comprises one or more of Y, La, Gd, Tb and Lu, such as in embodiments one or more of La and Y.

In embodiments, the luminescent material may alternatively or additionally comprise one or more of MS:Eu²⁺ and/or M₂Si₅N₈:Eu²⁺ and/or MAlSiN₃:Eu²⁺ and/or Ca₂AlSi₃O₂N₅:Eu²⁺, etc., wherein M comprises one or more of Ba, Sr and Ca, especially in embodiments at least Sr. Hence, in embodiments, the luminescent may comprise one or more materials selected from the group consisting of (Ba,Sr,Ca)S:Eu, (Ba,Sr,Ca)AlSiN₃:Eu and (Ba,Sr,Ca)₂Si₅N₈:Eu. In these compounds, europium (Eu) is substantially or only divalent, and replaces one or more of the indicated divalent cations. In general, Eu will not be present in amounts larger than 10% of the cation; its presence will especially be in the range of about 0.5 to 10%, more especially in the range of about 0.5 to 5% relative to the cation(s) it replaces. The term “:Eu”, indicates that part of the metal ions is replaced by Eu (in these examples by Eu²⁺). For instance, assuming 2% Eu in CaAlSiN₃:Eu, the correct formula could be (Ca_{0.98}Eu_{0.02})AlSiN₃. Divalent europium will in general replace divalent cations, such as the above divalent alkaline earth cations, especially Ca, Sr or Ba. The material (Ba,Sr,Ca)S:Eu can also be indicated as MS:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca). Further, the material (Ba,Sr,Ca)₂Si₅N₈:Eu can also be indicated as M₂Si₅N₈:Eu, wherein M is one or more elements selected from the group consisting of barium (Ba), strontium (Sr) and calcium (Ca); especially, M comprises in this compound Sr and/or Ba. In a further specific embodiment, M consists of Sr and/or Ba (not taking into account the presence of Eu), especially 50 to 100%, more especially 50 to 90% Ba and 50 to 0%, especially 50 to 10% Sr, such as Ba_{1.5}Sr_{0.5}Si₅N₈:Eu (i.e. 75% Ba; 25% Sr). Here, Eu is introduced and replaces at least part of M, i.e. one or more of Ba, Sr, and Ca). Likewise, the material (Ba,Sr,Ca)AlSiN₃:Eu can also be indicated as MAlSiN₃:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca). Eu in the above indicated luminescent materials is substantially or only in the divalent state, as is known to the person skilled in the art.

In embodiments, a red luminescent material may comprise one or more materials selected from the group consisting of (Ba,Sr,Ca)S:Eu, (Ba,Sr,Ca)AlSiN₃:Eu and (Ba,Sr,Ca)₂Si₅N₈:Eu. In these compounds, europium (Eu) is substantially or only divalent, and replaces one or more of the indicated divalent cations. In general, Eu will not be present in amounts larger than 10% of the cation; its presence will especially be in the range of about 0.5 to 10%, more especially in the range of about 0.5 to 5% relative to the cation(s) it replaces. The term “:Eu”, indicates that part of the metal ions is replaced by Eu (in these examples by Eu²⁺). For instance, assuming 2% Eu in CaAlSiN₃:Eu, the correct formula could be (Ca_{0.98}Eu_{0.02})AlSiN₃. Divalent europium will in general replace divalent cations, such as the above divalent alkaline earth cations, especially Ca, Sr or Ba.

The material (Ba,Sr,Ca)S:Eu can also be indicated as MS:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca).

Further, the material (Ba,Sr,Ca)₂Si₅N₈:Eu can also be indicated as M₂Si₅N₈:Eu, wherein M is one or more elements selected from the group consisting of barium (Ba), strontium (Sr) and calcium (Ca); especially, M comprises in this compound Sr and/or Ba. In a further specific embodiment, M consists of Sr and/or Ba (not taking into account the presence of Eu), especially 50 to 100%, more especially 50 to 90% Ba and 50 to 0%, especially 50 to 10% Sr, such as Ba_{1.5}Sr_{0.5}Si₅N₈:Eu (i.e. 75% Ba; 25% Sr). Here, Eu is introduced and replaces at least part of M, i.e. one or more of Ba, Sr, and Ca).

Likewise, the material (Ba,Sr,Ca)AlSiN₃:Eu can also be indicated as MAlSiN₃:Eu, wherein M is one or more elements selected from the group consisting of barium (Ba), strontium (Sr) and calcium (Ca); especially, M comprises in this compound calcium or strontium, or calcium and strontium, more especially calcium. Here, Eu is introduced and replaces at least part of M (i.e. one or more of Ba, Sr, and Ca).

Eu in the above indicated luminescent materials is substantially or only in the divalent state, as is known to the person skilled in the art.

Blue luminescent materials may comprise YSO (Y₂SiO₅:Ce³⁺), or similar compounds, or BAM (BaMgAl₁₀O₁₇:Eu²⁺), or similar compounds.

The term “luminescent material” herein especially relates to inorganic luminescent materials.

Alternatively or additionally, also other luminescent materials may be applied. For instance quantum dots and/or organic dyes may be applied and may optionally be embedded in transmissive matrices like e.g. polymers, like PMMA, or polysiloxanes, etc, etc. Quantum dots are small crystals of semiconducting material generally having a width or diameter of only a few nanometers. When excited by incident light, a quantum dot emits light of a color determined by the size and material of the crystal. Light of a particular color can therefore be produced by adapting the size of the dots. Most known quantum dots with emission in the visible range are based on cadmium selenide (CdSe) with a shell such as cadmium sulfide (CdS) and zinc sulfide (ZnS). Cadmium free quantum dots such as indium phosphide (InP), and copper indium sulfide (CuInS₂) and/or silver indium sulfide (AgInS₂) can also be used. Quantum dots show very narrow emission band and thus they show saturated colors. Furthermore the emission color can easily be tuned by adapting the size of the quantum dots. Any type of quantum dot known in the art may be used in the present invention. However, it may be preferred for reasons of environmental safety and concern to use cadmium-free quantum dots or at least quantum dots having a very low cadmium content.

Instead of quantum dots or in addition to quantum dots, also other quantum confinement structures may be used. The term “quantum confinement structures” should, in the context of the present application, be understood as e.g. quantum wells, quantum dots, quantum rods, tripods, tetrapods, or nanowires, etcetera.

Organic phosphors can be used as well. Examples of suitable organic phosphor materials are organic luminescent materials based on perylene derivatives, for example compounds sold under the name Lumogen® by BASF. Examples of suitable compounds include, but are not limited to, Lumogen® Red F305, Lumogen® Orange F240, Lumogen® Yellow F083, and Lumogen® F170.

Different luminescent materials may have different spectral power distributions of the respective luminescent material light. Alternatively or additionally, such different luminescent materials may especially have different color points (or dominant wavelengths).

As indicated above, other luminescent materials may also be possible. Hence, in specific embodiments the luminescent material is selected from the group of divalent europium containing nitrides (“nitride”), divalent europium containing oxynitrides (“oxynitride”), divalent europium containing silicates, cerium comprising garnets, and quantum structures. Quantum structures may e.g. comprise quantum dots or quantum rods (or other quantum type particles) (see above). Quantum structures may also comprise quantum wells. Quantum structures may also comprise photonic crystals.

As can be derived from the above, the term “different luminescent materials” may refer to luminescent materials that are different, or to two compositions, each including at least one luminescent material in common, but wherein the compositions differ. For instance, a first luminescent material comprising luminescent materials A and B, and a second luminescent material comprising only A or only B, or comprising both A and B, but in a different weight ratio. Such first luminescent material and second luminescent material may have different spectral power distributions of their respective luminescent material light.

In embodiments, the luminescent material may comprise a luminescent material of the type M′ₓM₂₋₂ₓAX₆ doped with tetravalent manganese, wherein M′ comprises an alkaline earth cation, M comprises an alkaline cation, and x is in the range of 0-1, wherein A comprises a tetravalent cation, for instance comprising one or more of silicon and titanium, wherein X comprises a monovalent anion, at least comprising fluorine. Such luminescent materials may herein also be indicated as “KSiF” or “KSF”, whether or not M comprises K or one or more other alkaline cations.

A luminescent material of the type M′ₓM₂₋₂ₓAX₆ doped with tetravalent manganese is amongst others described in WO2013121355A1, which is herein incorporated by reference. Passages from WO2013121355Al are also copied herein.

Herein, M′ₓM₂₋₂ₓAX₆ doped with tetravalent manganese, may further also shortly be indicated as “phosphor”, i.e. the phrase “phosphor comprising M′ₓM₂₋₂ₓAX₆ doped with tetravalent manganese” may in an embodiment also be read as M′ₓM₂₋₂ₓAX₆ doped with tetravalent manganese phosphor, or (tetravalent) Mn-doped M′ₓM₂₋₂ₓAX₆ phosphor, or shortly “phosphor”.

Relevant alkaline cations (M) are sodium (Na), potassium (K) and rubidium (Rb). Optionally, also lithium and/or cesium may be applied. In a preferred embodiment, M comprises at least potassium. In yet another embodiment, M comprises at least rubidium. The phrase “wherein M comprises at least potassium” indicates for instance that of all M cations in a mole M′ₓM₂₋₂ₓAX₆, a fraction comprises K⁺ and an optionally remaining fraction comprises one or more other monovalent (alkaline) cations (see also below). In another preferred embodiment, M comprises at least potassium and rubidium. Optionally, the M′ₓM₂₋₂ₓAX₆ luminescent material has the hexagonal phase. In yet another embodiment, the M′ₓM₂₋₂ₓAX₆ luminescent material has the cubic phase.

Relevant alkaline earth cations (M′) are magnesium (Mg), strontium (Sr), calcium (Ca) and barium (Ba), especially one or more of Sr and Ba.

In an embodiment, a combination of different alkaline cations may be applied. In yet another embodiment, a combination of different alkaline earth cations may be applied. In yet another embodiment, a combination of one or more alkaline cations and one or more alkaline earth cations may be applied. For instance, KRb_{0.5}Sr_{0.25}AX₆ might be applied. As indicated above, x may be in the range of 0-1, especially x<1. In an embodiment, x=0.

The term “tetravalent manganese” refers to Mn⁴⁺. This is a well-known luminescent ion. In the formula as indicated above, part of the tetravalent cation A (such as Si) is being replaced by manganese. Hence, M′ₓM₂₋₂ₓAX₆ doped with tetravalent manganese may also be indicated as M′ₓM₂₋₂ₓA₁₋ₘMnₘX₆. The mole percentage of manganese, i.e. the percentage it replaces the tetravalent cation A will in general be in the range of 0.1-15%, especially 1-12%, i.e. m is in the range of 0.001-0.15, especially in the range of 0.01-0.12.

A comprises a tetravalent cation, and preferably at least comprises silicon. A may optionally (further) comprise one or more of titanium (Ti), germanium (Ge), stannum (Sn) and zinc (Zn). Preferably, at least 80%, even more preferably at least 90%, such as at least 95% of M consists of silicon. Hence, in a specific embodiment, M′ₓM₂₋₂ₓAX₆ may also be described as M′ₓM₂₋₂ₓA_{1-m-t-g-s-zr}MnₘTiₜGe_{g}SnₛZr_{zr}X₆, wherein m and x are as indicated above, and wherein t,g,s,zr are each individually preferably in the range of 0-0.2, especially 0-0.1, even more especially 0-0.05, wherein t+g+s+zr is smaller than 1, especially equal to or smaller than 0.2, preferably in the range of 0-0.2, especially 0-0.1, even more especially 0-0.05, and wherein A is especially Si. X is preferably fluorine (F).

As indicated above, M relates to monovalent cations, but preferably at least comprises potassium and/or rubidium. Other monovalent cations that may further be comprised by M can be selected from the group consisting of lithium (Li), sodium (Na), cesium (Cs) and ammonium (NH⁴⁺). In an embodiment, preferably at least 80% (i.e. 80% of all moles of the type M), even more preferably at least 90%, such as 95% of M consists of potassium and/or rubidium. Especially, in these embodiments x is thus zero.

Hence, in a specific embodiment, M′ₓM₂₋₂ₓAX₆ can also be described as (K_{1-r-l-n-c-nh}RbᵣLiₗNaₙCs_{c}(NH₄)ₙₕ)₂AX₆, wherein r is in the range of 0-1, wherein l,n,c,nh are each individually preferably in the range of 0-1, preferably 0-0.2, especially 0-0.1, even more especially 0-0.05, and wherein r+l+n+c+nh is in the range of 0-1, especially 1+n+c+nh is smaller than 1, especially equal to or smaller than 0.2, preferably in the range of 0-0.2, especially 0-0.1, even more especially 0-0.05. X is preferably fluorine (F).

As indicated above, instead of or in addition to the alkaline cation(s), also one or more alkaline earth cations may be present. Hence, in a specific embodiment, M′ₓM₂₋₂ₓAX₆ can also be described as Mg_{mg}Ca_{ca}SrₛᵣBa_{ba}(KₖRbᵣLiₗNaₙC_{Sc}(NH₄)ₙₕ)₂AX₆, with k, r, l, n, c, nh each individually being in the range of 0-1, wherein mg, ca, sr, ba are each individually in the range of 0-1, and wherein mg+ca+sr+ba+k+r+l+n+c+nh=1. In embodiments, k=1, and the others (mg, ca, sr, ba, r, l, n, c, nh) are zero.

As indicated above, X relates to a monovalent anion, but at least comprises fluorine. Other monovalent anions that may optionally be present may be selected from the group consisting of chlorine (Cl), bromine (Br), and iodine (I). Preferably, at least 80%, even more preferably at least 90%, such as 95% of X consists of fluorine. Hence, in a specific embodiment, M′ₓM₂₋₂ₓAX₆ can also be described as M′ₓM₂₋₂ₓA(F_{1-cl-b-i}Cl_{cl}Br_{b}Iᵢ)₆, wherein cl,b,i are each individually preferably in the range of 0-0.2, especially 0-0.1, even more especially 0-0.05, and wherein cl+b+i is smaller than 1, especially equal to or smaller than 0.2, preferably in the range of 0-0.2, especially 0-0.1, even more especially 0-0.05. Especially, X essentially consists of F (fluorine).

Hence, M′ₓM₂₋₂ₓAX₆ can also be described as (K_{1-r-l-n-c-nh}RbᵣLiₗNaₙC_{Sc}(NH₄)ₙₕ)₂Si_{1-m-t-g-s-zr}MnₘTiₜGe_{g}SnₛZr_{zr}(F_{1-cl-b-i}Cl_{cl}Br_{b}Iᵢ)₆, with the values for r,l,n,c,nh,m,t,g,s,zr,cl,b,i as indicated above. X is preferably fluorine (F).

Even more especially, M′ₓM₂₋₂ₓAX₆ can also be described as Mg_{mg}Ca_{ca}SrₛᵣBa_{ba}(KₖRbᵣLiₗNaₙCs_{c}(NH₄)ₙₕ)₂Si_{1-m-t-g-s-zr}MnₘTiₜGe_{g}SnₛZr_{zr}(F_{1-cl-b-i}Cl_{cl}Br_{b}Iᵢ)₆, with k, r, l, n, c, nh each individually being in the range of 0-1, wherein mg, ca, sr, ba are each individually in the range of 0-1, wherein mg+ca+sr+ba+k+r+l+n+c+nh=1, and with the values for m,t,g,s,zr,cl,b,i as indicated above. X is preferably fluorine (F).

In an embodiment, M′ₓM₂₋₂ₓAX₆ comprises K₂SiF₆ (indicated herein also as KSiF system). As indicated above, in another preferred embodiment, M′ₓM₂₋₂ₓAX₆ comprises KRbSiF₆ (i.e. r=0.5 and l,n,c,nh,t,g,s,zr,cl,b,i are 0) (herein also indicated as K,Rb system). As indicated above, part of silicon is replaced by manganese (i.e. the formula may also be described as K₂Si₁₋ₘMnₘF₆ or KRbSi₁₋ₘMnₘF₆, with m as indicated above, or as KRbSiF₆:Mn and K₂SiF₆:Mn, respectively). As manganese replaces part of a host lattice ion and has a specific function, it is also indicated as “dopant” or “activator”. Hence, the hexafluorosilicate is doped or activated with manganese (Mn⁴⁺).

In specific embodiments, the luminescent material may comprise (K,Rb)₂SiF₆:Mn⁴⁺. Alternatively or additionally, in embodiments the third luminescent material may comprise K₂SiF₆:Mn⁴⁺. Alternatively or additionally, in embodiments the third luminescent material may comprise K₂TiF₆:Mn⁴⁺. In embodiments, the third luminescent material may comprise K₂(Si,Ti)F₆:Mn⁴⁺. As can be derived from the above, “Si, Ti” may indicate one or more of Si and Ti.

The luminescent material may also be coated, as also described in WO2013121355A1.

Here below, M′ₓM₂₋₂ₓAX₆ is also indicated as M′ₓM″₂₋₂ₓAX₆. In specific embodiments, the indication M′ₓM₂₋₂ₓAX₆ may refer to one or more of (K,Rb)₂SiF₆:Mn⁴⁺, (K,Rb)₂TiF₆:Mn⁴⁺, K₂(Si,Ti)F₆:Mn⁴⁺, and Rb₂(Si,Ti)F₆:Mn⁴⁺, such as one or more of K₂TiF₆:Mn⁴⁺, of K₂SiF₆:Mn⁴⁺, and of Rb₂SiF₆:Mn⁴⁺.

Herein, the term “luminescent material light” may refer to the light generated by a luminescent material (including a combination of different luminescent materials). The term “luminescent convert light” (or “converter light”) may refer to the light generated by the luminescent converter. Essentially, this luminescent converter light may consist of luminescent material light.

Returning to the light generating devices, first some embodiments in relation to the first light generating device are described and then some embodiments in relation to the second light generating device are described.

Especially, the first light source may comprise a solid state light source. Further, the first light source may be configured to generate first light source light having a first light source centroid wavelength (λ_{c,S1}) selected from the wavelength range of 380-420 nm. Especially, this light source light may be used to provide luminescent converter light via the first luminescent material, but this light source light may also be used to provide light that may have a disinfection function and/or provides a visible effect.

In specific embodiments, the first light source centroid wavelength (λ_{c,S1}) is selected from the range of 400-420 nm. This may provide a disinfection function and/or may provide a (better) visual effect (than would the first light source centroid wavelength (λ_{c,S1}) be selected from the range of 380-400 nm). Further, this may be light that may be relatively well converted by a number of luminescent materials, such as described herein.

Especially the first luminescent converter may be configured to convert at least part of the first light source light into first luminescent converter light having a first luminescent converter centroid wavelength (λ_{c,L1}) selected from the wavelength range of 590-780 nm. Therefore, especially the first luminescent converter light may comprise orange and/or red light (see also below). Further, especially the luminescent material may not be used as such, but comprised by a matrix material (see also above). Especially, in embodiments the luminescent material may be dispersed in the matrix material. The matrix material may e.g. be a silicone or an organic polymeric material. The matrix material may in embodiments be a cross-linked organic material, such as a cross-linked polymeric material; see further also below. Hence, in embodiment the first luminescent converter may comprise a first matrix material and a first luminescent material, wherein the first luminescent material has a first weight percentage CW₁ relative to a total weight of the first luminescent converter. In embodiments, CW₁ may be selected from the range of 2-50 wt %, such as selected from the range 2-49.8 wt %, such as selected from the range 2-47.5 wt %, like selected from the range of 2-30 wt. %, such as selected from the range of 2-20 wt %. Note that the term “luminescent material” may also refer to a plurality of luminescent materials. Then, CW₁ refers to the combination of the luminescent materials.

The term “xyz centroid wavelength”, such as “first light source centroid wavelength” especially refers to the centroid wavelength of xyz light, such as of the first light source light. Likewise, this applies to similar terms. For the sake of completeness, the term “xyz centroid wavelength”, such as “first luminescent converter centroid wavelength” especially refers to the centroid wavelength of xyz light, such as of the first luminescent converter light. Likewise, this applies to similar terms.

In specific embodiment, the first light generating device may be configured to generate first device light having a spectral power distribution in the wavelength range of 380-780 nm with at least 55%, more especially at least 60%, of a spectral power provided by the first light source light and at maximum 45%, more especially at maximum 40%, of the spectral power provided by the first converter light. Further, especially the first light generating device may be configured to generate first device light having a spectral power distribution in the wavelength range of 380-780 nm with at maximum 95%, more especially at maximum 90%, of a spectral power provided by the first light source light and at minimum 5%, more especially at minimum 10%, of the spectral power provided by the first converter light. Especially, in embodiments the first device light may have a spectral power distribution in the wavelength range of 380-780 nm with at maximum 85% of a spectral power provided by the first light source light and at minimum 15% of the spectral power provided by the first converter light. In this way, the first device light may in embodiments be violet light, due to the contribution of the first light source light and the first luminescent converter light.

For instance, in embodiments the first device light may have a spectral power distribution in the wavelength range of 380-780 nm with at maximum 60% of the spectral power provided by the first converter light, more especially at maximum 50% of the spectral power provided by the first converter light, such as at maximum 40% of the spectral provided by the first converter light. Yet, in embodiments the first device light may have a spectral power distribution in the wavelength range of 380-780 nm with at minimum 40% of the spectral power provided by the first light source light, more especially at minimum 50% of the spectral power provided by the first light source light, such as at minimum 60% of the spectral power provided by the first light source light.

Further, especially the second light source may comprise a solid state light source. Further, the second light source may be configured to generate second light source light having a second light source centroid wavelength (λ_{c,S2}) selected from the wavelength range of 430-490 nm. Especially, this light source light may be used to provide luminescent converter light via the second luminescent material, but this light source light may also be used to provide light in the visible, which, together with the device light, may in embodiments be white light or off-white light (see also below). Further, in embodiments the second light source centroid wavelength (λ_{c,S2}) may be selected from the range of 430-470 nm, more especially 440-470 nm. This may provide the blue light necessary for the white light and may also be well converted by a number of luminescent materials as described herein.

Especially the second luminescent converter may be configured to convert at least part of the second light source light into second converter light having a second luminescent converter centroid wavelength (λ_{c,L2}) selected from the wavelength range of 490-780 nm. Therefore, especially the second luminescent converter light may comprise one or more of green, yellow, orange, and red light (see also below). Further, especially the luminescent material may not be used as such, but comprised by a matrix material (see also above). Especially, in embodiments the luminescent material may be dispersed in the matrix material. The matrix material may, e.g., be a silicone or an organic polymeric material. The matrix material may in embodiments be a cross-linked organic material, such as a cross-linked polymeric material; see further also below. Hence, in embodiments the second luminescent converter comprises a second matrix material and a second luminescent material, wherein the second luminescent material has a second weight percentage CW₂ relative to a total weight of the second luminescent converter.

In embodiments, CW₁ may be selected from the range of 4-99.6 wt %, such as selected from the range of 4-95, like selected from the range of 4-60 wt %, such as selected from the range of 4-40 wt %. Note that the term “luminescent material” may also refer to a plurality of luminescent materials. Then, CW₂ refers to the combination of the luminescent materials.

In specific embodiments, the second light generating device is configured to generate second device light having a spectral power distribution in the wavelength range of 380-780 nm with at least 55%, more especially at least 60% of the spectral power provided by the second converter light and at maximum 45%, more especially at maximum 40% of the spectral power provided by the second light source light. Further, especially the second light generating device may be configured to generate second device light having a spectral power distribution in the wavelength range of 380-780 nm with at maximum 90%, more especially at maximum 85% of the spectral power provided by the second converter light and at minimum 5%, more especially at minimum 10% of the spectral power provided by the second light source light. Especially, in embodiments the second device light may have a spectral power distribution in the wavelength range of 380-780 nm with at maximum 85% of the spectral power provided by the second converter light and at minimum 15% of the spectral power provided by the second light source light.

For instance, in embodiments the second device light may have a spectral power distribution in the wavelength range of 380-780 nm with at maximum 60% of the spectral power provided by the second light source light, more especially at maximum 50% of the spectral power provided by the second light source light, such as at maximum 40% of the spectral provided by the second light source light. Yet, in embodiments the second device light may have a spectral power distribution in the wavelength range of 380-780 nm with at minimum 40% of the spectral power provided by the second converter light, more especially at minimum 50% of the spectral power provided by the second converter light, such as at minimum 60% of the spectral power provided by the second converter light.

Note that the first luminescent converter centroid wavelength may be the centroid wavelength of the (first) luminescent converter light which may consist of a contribution of a single type of luminescent material, or which may consist of two or more contributions of two or more different types of luminescent materials. Likewise, the second luminescent converter centroid wavelength may be the centroid wavelength of the (second) luminescent converter light which may consist of a contribution of a single type of luminescent material, or which may especially consist of two or more contributions of two or more different types of luminescent materials. See further also below.

Especially, in embodiments CW₁/CW₂≤0.5 may apply. In other words, the weight percentage of luminescent material in the first luminescent converter is lower than the weight percentage of the luminescent material in the second luminescent converter. As indicated above, this may have as advantage that quenching or reabsorption in the first luminescent converter may be reduced. Especially, in embodiments 0.02≤CW₁/CW₂≤0.5 (may apply), such as 0.05≤CW₁/CW₂≤0.5, like in embodiments 0.05≤CW₁/CW₂≤0.45. Too low values may provide system light with a relatively low disinfection and/or optical function.

Especially, the light generating system may be configured to generate system light. The system light may comprise one or more of the first device light and the second device light. In embodiments wherein the spectral power distribution of the system light may not be controllable, the system light may comprise both the first device light and the second device light. Hence, in embodiments the system light may be controllable.

The term “controlling” and similar terms especially refer at least to determining the behavior or supervising the running of an element. Hence, herein “controlling” and similar terms may e.g. refer to imposing behavior to the element (determining the behavior or supervising the running of an element), etc., such as e.g. measuring, displaying, actuating, opening, shifting, changing temperature, etc., Beyond that, the term “controlling” and similar terms may additionally include monitoring. Hence, the term “controlling” and similar terms may include imposing behavior on an element and also imposing behavior on an element and monitoring the element. The controlling of the element can be done with a control system, which may also be indicated as “controller”. The control system and the element may thus at least temporarily, or permanently, functionally be coupled. The element may comprise the control system. In embodiments, the control system and element may not be physically coupled. Control can be done via wired and/or wireless control. The term “control system” may also refer to a plurality of different control systems, which especially are functionally coupled, and of which e.g. one control system may be a master control system and one or more others may be slave control systems. A control system may comprise or may be functionally coupled to a user interface.

The control system may also be configured to receive and execute instructions from a remote control. In embodiments, the control system may be controlled via an App on a device, such as a portable device, like a Smartphone or I-phone, a tablet, etc., The device is thus not necessarily coupled to the lighting system, but may be (temporarily) functionally coupled to the lighting system.

Hence, in embodiments the control system may (also) be configured to be controlled by an App on a remote device. In such embodiments the control system of the lighting system may be a slave control system or control in a slave mode. For instance, the lighting system may be identifiable with a code, especially a unique code for the respective lighting system. The control system of the lighting system may be configured to be controlled by an external control system which has access to the lighting system on the basis of knowledge (input by a user interface of with an optical sensor (e.g. QR code reader) of the (unique) code. The lighting system may also comprise means for communicating with other systems or devices, such as on the basis of Bluetooth, Thread, WIFI, LiFi, ZigBee, BLE or WiMAX, or another wireless technology.

The system, or apparatus, or device may execute an action in a “mode” or “operation mode” or “mode of operation” or “operational mode”. The term “operational mode may also be indicated as “controlling mode”. Likewise, in a method an action or stage, or step may be executed in a “mode” or “operation mode” or “mode of operation” or “operational mode”. This does not exclude that the system, or apparatus, or device may also be adapted for providing another controlling mode, or a plurality of other controlling modes. Likewise, this may not exclude that before executing the mode and/or after executing the mode one or more other modes may be executed.

However, in embodiments a control system may be available, that is adapted to provide at least the controlling mode. Would other modes be available, the choice of such modes may especially be executed via a user interface, though other options, like executing a mode in dependence of a sensor signal or a (time) scheme, may also be possible. The operation mode may in embodiments also refer to a system, or apparatus, or device, that can only operate in a single operation mode (i.e. “on”, without further tunability).

Hence, in embodiments, the control system may control in dependence of one or more of an input signal of a user interface, a sensor signal (of a sensor), and a timer. The term “timer” may refer to a clock and/or a predetermined time scheme.

In specific embodiments, the light generating system may be configured to provide in an operational mode white system light. The term “white light”, and similar terms, herein, is known to the person skilled in the art. It may especially relate to light having a correlated color temperature (CCT) between about 1800 K and 20000 K, such as between 2000 and 20000 K, especially 2700-20000 K, for general lighting especially in the range of about 2000-7000 K, such as in the range of 2700 K and 6500 K. In embodiments, e.g. for backlighting purposes, or for other purposes, the correlated color temperature (CCT) may especially be in the range of about 7000 K and 20000 K. Yet further, in embodiments the correlated color temperature (CCT) is especially within about 15 SDCM (standard deviation of color matching) from the BBL (black body locus), especially within about 10 SDCM from the BBL, even more especially within about 5 SDCM from the BBL. More especially, the light generating devices may be selected such to provide the system light wherein the color rendering index is at least 65, more especially at least 70, such as at least 80, and/or the correlated color temperature may be selected from the range of 1800-8000 K, more especially 1800-6500 K.

In embodiments, wherein the system light is white light, a contribution to the spectral power in the wavelength range of 380-780 nm by the first device light may be selected from the range of about 10-70% (such as especially 15-45%), and a contribution to the spectral power in the wavelength range of 380-780 nm by the second device light may be selected from the range of about 30-98% (such as especially 55-85%).

As indicate above, the luminescent material comprised by the first light generating device may especially emit in the orange-red wavelength range, and have a centroid wavelength in the about orange-red wavelength range. Above, several possible types of luminescent materials have been described. Especially, in embodiments the first luminescent material may comprise one or more of a Eu²⁺-based luminescent material and a Mn⁴⁺-based luminescent material, and wherein the second luminescent material may comprise one or more of a Eu²⁺-based luminescent material and a Mn⁴⁺-based luminescent material. More especially, in embodiments the first luminescent material and the second luminescent material comprise one or more luminescent materials individually selected from the group MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese, wherein M comprises one or more of Ba, Sr, and Ca, especially in embodiments at least Sr; wherein M′ comprises an alkaline earth cation, wherein M″ comprises a cation, and x is in the range of 0-1, wherein A comprises a tetravalent cation, wherein X comprises a monovalent anion, at least comprising fluorine; and wherein the first luminescent material and the second luminescent material differ in composition.

Especially, the first luminescent material and the second luminescent material may differ. When the first luminescent material comprises a combination of two or more luminescent materials, and/or when the second luminescent material comprises a combination of two or more luminescent materials, even then the first luminescent material and the second luminescent material may differ. In other words, the first luminescent material and the second luminescent material may comprise different luminescent material compositions. Hence, essentially, in embodiments the first luminescent converter light and the second luminescent converter light may have different spectral power distributions. As indicated above, the former may have a color point in about the orange-red, and the latter may have a color point in or closer to the white. In specific embodiments, colors or color points of a first type of light and a second type of light may be different when the respective color points of the first type of light and the second type of light differ with at least 0.01 for u′ and/or with at least 0.01 for v′, even more especially at least 0.02 for u′ and/or with at least 0.02 for v′. In yet more specific embodiments, the respective color points of first type of light and the second type of light may differ with at least 0.03 for u′ and/or with at least 0.03 for v′. Here, u′ and v′ are color coordinates of the light in the CIE 1976 UCS (uniform chromaticity scale) diagram.

Hence, in embodiments (a) the first luminescent material may comprise one or more of MS:Eu²⁺, M₂Si₅N₈:Eu²⁺, MAlSiN₃:Eu²⁺, Ca₂AlSi₃O₂N₅:Eu²⁺, and (b) the second luminescent material may comprise one or more of M′ₓM″₂₋₂ₓAX₆ doped with tetravalent manganese. This may provide white light with a relatively high CRI. More especially in embodiments less than 10% wt. % of the first luminescent material may be provided by M′ₓM″₂₋₂ₓAX₆, and/or less than 10% wt. % of the second luminescent material may be provided by one or more of MS:Eu²⁺, M₂Si₅N₈:Eu²⁺, MAlSiN₃:Eu²⁺, Ca₂AlSi₃O₂N₅:Eu²⁺. Especially, in embodiments (a) the second luminescent material does not comprise one or more of MS:Eu²⁺, M₂Si₅No:Eu²⁺, MAlSiN₃:Eu²⁺, Ca₂AlSi₃O₂N₅:Eu²⁺, and (b) the first luminescent material does not comprise one or more of M′ₓM″₂₋₂ₓAX₆ doped with tetravalent manganese. Such embodiments may be beneficial for preventing or reducing cross-talk. Further, also a higher difference between the refractive index of the matrix material and the luminescent material may be obtained.

In other embodiments (a) the second luminescent material may comprise one or more of MS:Eu²⁺, M₂Si₅N₈:Eu²⁺, MAlSiN₃:Eu²⁺, Ca₂AlSi₃O₂N₅:Eu²⁺, and (b) the first luminescent material may comprise M′ₓM″₂₋₂ₓAX₆ doped with tetravalent manganese. This may be a (slightly) more energy efficient solution and/or may allow a (further) variation of the CRI. More especially in embodiments less than 10% wt. % of the second luminescent material may be provided by M′ₓM″₂₋₂ₓAX₆, and/or less than 10% wt. % of the first luminescent material may be provided by one or more of MS:Eu²⁺, M₂Si₅N₈:Eu²⁺, MAlSiN₃:Eu²⁺, Ca₂AlSi₃O₂N₅:Eu²⁺. Especially, in embodiments (a) the first luminescent material does not comprise one or more of MS:Eu²⁺, M₂Si₅N₈:Eu²⁺, MAlSiN₃:Eu²⁺, Ca₂AlSi₃O₂N₅:Eu²⁺, and (b) the second luminescent material does not comprise one or more of M′ₓM″₂₋₂ₓAX₆ doped with tetravalent manganese. Such embodiments may be beneficial for preventing or reducing cross-talk. Further, also a higher difference between the refractive index of the matrix material and the luminescent material may be obtained.

In (other) embodiments, the first luminescent material may comprise two or more types of different luminescent materials and/or the second luminescent material may comprise two or more types of different luminescent materials. The spectral power distributions of the respective first luminescent converter light and second luminescent converter light, however, differ, as the relative contributions and/or the type of luminescent materials comprised by the first luminescent converter and second luminescent converter may differ.

In embodiments, the first luminescent converter may comprise (a) an oxynitride luminescent material and/or a nitride luminescent material, and (b) a M′ₓM″₂₋₂ₓAX₆ doped with tetravalent manganese luminescent material.

Yet, in embodiments, the first luminescent material and/or the second luminescent material may each comprise two or three different red luminescent materials e.g. selected from KSiF, oxynitride luminescent material and nitride luminescent material.

In embodiments the first luminescent material may consist of luminescent materials that have centroid wavelengths of their respective luminescent material light in the orange-red wavelength range. In specific embodiments, the first luminescent material may essentially consist of one or more of MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese.

In embodiments, the first luminescent material may comprise at least 60 wt % of M′ₓM″₂₋₂ₓAX₆ doped with tetravalent manganese, and between 5-40 wt % of one or more other luminescent materials, especially selected from oxynitride luminescent material and nitride luminescent material. In other embodiments, the first luminescent material comprises (a) at least 60 wt % of one or more other luminescent materials, especially selected from oxynitride luminescent material and nitride luminescent material and (b) between 5-40 wt % of M′ₓM″₂₋₂ₓAX₆ doped with tetravalent manganese.

In specific embodiments, the first luminescent material may comprise at least 70 wt % of M′ₓM″₂₋₂ₓAX₆ doped with tetravalent manganese, and between 5-30 wt % of one or more other luminescent materials, especially selected from oxynitride luminescent material and nitride luminescent material. In other specific embodiments, the first luminescent material comprises (a) at least 70 wt % of one or more other luminescent materials, especially selected from oxynitride luminescent material and nitride luminescent material and (b) between 5-30 wt % of M′M″₂₋₂ₓAX₆ doped with tetravalent manganese.

In embodiments, the second luminescent material may (also) comprise one or more luminescent materials having a centroid wavelength in the green-yellow wavelength range. Especially, in embodiments the second luminescent converter may comprise one or more second luminescent materials of the type A3B5O12:Ce, wherein A in embodiments comprises one or more of Y, La, Gd, Tb and Lu, especially (at least) one or more of Y, Gd, Tb and Lu, and wherein B in embodiments comprises one or more of Al, Ga, In and Sc.

In specific embodiments, the second luminescent converter may comprise at least two second luminescent materials of the type A₃B5O12:Ce, wherein a first second luminescent material of this type may comprise no Lu or may comprise more Y than Lu (on a molar basis), and wherein a second luminescent material of this type may comprise no Y or may comprise more Lu than Y (on a molar basis). In embodiments, the first second luminescent material of this type A (of A3B5O12:Ce) may comprise Y, Lu, and Ce, wherein Ce/Y>0 (see also above for the mole percentages of Ce), and wherein 0≤Lu/Y≤0.05. Note that this may include embodiments wherein no Lu is available. Alternatively or additionally, the first second luminescent material of this type A (of A3B5O12:Ce) may comprise Y, Lu, and Ce, wherein Ce/Lu>0 (see also above for the mole percentages of Ce), and wherein 0≤Y/Lu≤0.9. Note that this may include embodiments wherein no Y is available. In such embodiments, the CRI of the second converter light, and especially of the system light, may be relatively high. The ratios are on molar basis.

In embodiments, the second luminescent material may comprise (a) one or more second luminescent materials of the type A₃B5O12:Ce³⁺, and (b) one or more second luminescent materials selected from the group of MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese. Yet, in embodiments the second luminescent material may comprise (a) two or more different types of A₃B5O12:Ce³⁺, and one or more of more second luminescent materials of the type A₃B5O12:Ce³⁺, and (b) one or more of second luminescent materials selected from the group of MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese.

In yet further specific embodiments, the second luminescent material comprises one or more of M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese. Yet, in embodiments the second luminescent material comprises (a) one or more of M₂Si₅N₈:Eu²⁺, MAlSiN₃:Eu²⁺, Ca₂AlSi₃O₂N₅:Eu²⁺, and (b) M′ₓM″₂₋₂ₓAX₆ doped with tetravalent manganese. In such embodiments, the CRI of the second converter light, and especially of the system light, may be relatively high.

In embodiments, the second luminescent material may comprise on a weight basis at least 5 wt % points more of M′ₓM″₂₋₂ₓAX₆ doped with tetravalent manganese, than any other second luminescent material selected from MS:Eu²⁺, M₂Si₅N₈:Eu²⁺, MAlSiN₃:Eu²⁺, Ca₂AlSi₃O₂N₅:Eu²⁺, such as at least 10 wt % points. Note that this may imply that in specific embodiments none of MS:Eu²⁺, M₂Si₅N₈:Eu²⁺, MAlSiN₃:Eu²⁺, Ca₂AlSi₃O₂N₅:Eu²⁺ is comprised by the second luminescent material. In other embodiments, the second luminescent material may comprise on a weight basis at least 5 wt % points more of one or more second luminescent materials selected from MS:Eu²⁺, M₂Si₅N₈:Eu²⁺, MAlSiN₃:Eu²⁺, Ca₂AlSi₃O₂N₅:Eu²⁺, than M′ₓM″₂₋₂ₓAX₆ doped with tetravalent manganese. Note that this may imply that in specific embodiments M′ₓM″₂₋₂ₓAX₆ doped with tetravalent manganese is not comprised by the second luminescent material.

Hence, in embodiments, the second luminescent material may comprise luminescent materials that have centroid wavelengths of their respective luminescent material light in the orange-red wavelength range, such as one or more of MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese, especially in combination with a luminescent materials of the type A₃B5O12:Ce³⁺. Note that the term “a luminescent materials”, and similar terms, may in embodiments refer to a plurality (of different luminescent materials).

Alternatively or additionally, the luminescent material may comprise a luminescent material of the type A₃Si₆N₁₁:Ce³⁺, wherein A comprises one or more of Y, La, Gd, Tb and Lu, such as in embodiments one or more of La and Y. In specific embodiments, alternative to or additional to a garnet luminescent material, the luminescent material may comprise a luminescent material of the type A₃Si₆N₁₁:Ce³⁺, wherein A comprises one or more of Y, La, Gd, Tb and Lu, such as in embodiments one or more of La and Y.

As indicated above, the luminescent material may be comprised by a luminescent converter, wherein the luminescent converter further comprises a matrix material. As indicated above, the luminescent material may be dispersed (e.g. as particulate material) in the matrix material. The first matrix material and the second matrix material may comprise the same type of materials or may comprise different materials. In embodiments, the first matrix material and the second matrix material may individually be selected from an organic polymeric material, a silicone, and a (non-luminescent) silicate, and may in specific embodiments comprise cross-linked poly dimethyl siloxane. The matrix material may in embodiments comprise a resin.

Especially, the first matrix material and the second matrix material may essentially be non-luminescent. This may imply that a contribution of luminescence from the matrix material may be less than 1% to the total spectral power distribution in the 380-780 nm wavelength range, such as not larger than 0.1%, like essentially no luminescence. The term (non-luminescent) silicate is used as there are also divalent europium comprising silicates known. In general, such silicates may not be used as host matrix, but e.g. water glass (sodium silicate) as matrix material may be used.

As indicated above, in embodiments the second device light may be white light, or whitish light, or off-white light. Anyhow, the spectral power distribution of the second device light may be chosen such that together with the first device light, in an operational mode of the light generating system the system light is white (system light) (and wherein more especially the light generating devices (110,120) may be selected such to provide the system light wherein the color rendering index (of the system light) may be at least 80 and the correlated color temperature may be selected from the range of 1800-6500 K). Hence, in specific embodiments the second light generating device may be configured to generate white second device light, wherein in further embodiments the color rendering index of the second device light may be lower than the color rendering index of the white system light. Yet, in embodiments the second light generating device is configured to generate second device light having a color point that is configured further away from the black body locus than the color point of the white system light. In further specific embodiments, the second light generating device may be configured to generate second device light having a color point of at least 15 Standard Deviation of Color Matching from the black body locus. The system light, when being white, may especially be within 10 SDCM from the BBL, more especially within 7 SDCM from the BBL. Therefore, in further specific embodiments, the second light generating device may be configured to generate second device light having a color point of at least 15 Standard Deviation of Color Matching from the black body locus, and (thus) not being within 10 SDCM from the BBL, more especially not being within 7 SDCM from the BBL, but being at a distance of 15 SDCM or more from the BBL. Especially, in embodiments the system light may comprise both the first device light and the second device light (in one or more operational modes of the light generating system).

Especially, the light generating system may thus comprise two different types of light generating devices. Further, especially, the first luminescent material is not configured downstream of the second light source and the second luminescent material is not configured downstream of the first light source. Hence, in embodiments the first light generating device and the second light generating device may be individually controllable.

The terms “upstream” and “downstream” relate to an arrangement of items or features relative to the propagation of the light from a light generating means (here the especially the light source), wherein relative to a first position within a beam of light from the light generating means, a second position in the beam of light closer to the light generating means is “upstream”, and a third position within the beam of light further away from the light generating means is “downstream”.

The system may comprise optics to combine the first device light and the second device light. Optionally, the system may comprise further light generating devices, different from the first light generating device and the second light generating device. In specific embodiments, the system light may only be provided by one or more first light generating devices and/or one or more second light generating devices.

The term “optics” may especially refer to (one or more) optical elements. Hence, the terms “optics” and “optical elements” may refer to the same items. The optics may include one or more or mirrors, reflectors, collimators, lenses, prisms, diffusers, phase plates, polarizers, diffractive elements, gratings, dichroics, arrays of one or more of the afore-mentioned, etc. Alternatively or additionally, the term “optics” may refer to a holographic element or a mixing rod. In embodiments, the optics may include one or more of beam expander optics and zoom lens optics. In embodiments, the optics may comprise an integrator, like a “Koehler integrator” (or “Köhler integrator”).

In embodiments, the optics may comprise light mixing optics. The light mixing optics may comprise one or more of diffusers (surface or volume scattering diffusers or engineered holographic optical elements), light pipes, light guides, Koehler integrator optics, etc. Alternatively or additionally, the light mixing optics may comprise a collimator or other collimating optics. Alternatively or additionally, the light mixing optics may comprise a dichroic beam combiner, such as in specific embodiments a dichroic cube. In embodiments, the light mixing optics may comprise a dichroic beam splitter.

The light generating system may be part of or may be applied in e.g. office lighting systems, household application systems, shop lighting systems, home lighting systems, accent lighting systems, spot lighting systems, theater lighting systems, fiber-optics application systems, projection systems, self-lit display systems, pixelated display systems, segmented display systems, warning sign systems, medical lighting application systems, indicator sign systems, decorative lighting systems, portable systems, automotive applications, (outdoor) road lighting systems, urban lighting systems, green house lighting systems, horticulture lighting, digital projection, or LCD backlighting. The light generating system (or luminaire) may be part of or may be applied in e.g. optical communication systems or disinfection systems.

With such system, it may be possible to provide light having a controllable spectral power distribution. Further, with such system it may be possible to provide light having a controllable correlated color temperature and/or a controllable color rendering index. Yet, with such system it may be possible to provide a spectral power distribution partially or substantially conformal to the spectral power distribution (in the visible) of a black body radiator (emission). The fact that the light generating system may be configured to generate (white) system light, does not exclude embodiments of the system, wherein the system may also be able to operate or be operated in other operational modes. Especially, in embodiments the spectral power distribution of the system light may be controllable. Hence, in embodiments in a first operational mode (of the light generating system), the light generating system may be configured to generate the white system light and in a second operational mode (of the light generating system) the light generating system may be configured to generate non-white system light.

Therefore, in embodiments the light generating system may further comprise a control system configured to control a spectral power distribution of the system light. Especially, the control system may be configured to control the correlated color temperature of the system light at a value selected from the range of 1800-12000 K, such as selected from the range of 1800-6500 K, though other values are herein not excluded (see also above). In specific embodiments, the correlated color temperature of the system light may be controllable over a CCT control range of at least 500 K within the range of 1800-12000 K, such as selected from the range of 1800-6500 K, such as controllable over a CCT control range of at least 1000 K. For instance, the CCT of the system light may controllable between 2700-4000 K (i.e. over a CCT control range of 1300 K), or over a range of 2000-4500 K (i.e. over a CCT control range of 2500 K). Hence, in embodiments, the CCT (of the white system light) may be controlled from a first value T1 to a second value T2, wherein |T2−T1|≥500 K, more especially |T2−T1|≥1000 K.

In yet a further aspect, the invention also provides a lamp or a luminaire comprising the light generating system as defined herein. The luminaire may further comprise a housing, optical elements, louvres, etc, etc., The lamp or luminaire may further comprise a housing enclosing the light generating system. The lamp or luminaire may comprise a light window in the housing or a housing opening, through which the system light may escape from the housing. In yet a further aspect, the invention also provides a projection device comprising the light generating system as defined herein. Especially, a projection device or “projector” or “image projector” may be an optical device that projects an image (or moving images) onto a surface, such as e.g. a projection screen. The projection device may include one or more light generating systems such as described herein. Hence, in an aspect the invention also provides a lighting device selected from the group of a lamp, a luminaire, a projector device, a disinfection device, a photochemical reactor, and an optical wireless communication device, comprising the light generating system as defined herein. The lighting device may comprise a housing or a carrier, configured to house or support, one or more elements of the light generating system. For instance, in embodiments the lighting device may comprise a housing or a carrier, configured to house or support one or more of the first light generating device and the second light generating device. As indicated above, the first light generating device and the second light generating device may share a same support (such as a PCB).

In yet a further aspect, the invention provides a method for treating at least part of a space or of an object, wherein the method comprises providing system light comprising the first device light in the space or to the object, using the light generating system or the lighting device as described herein.

The terms “visible”, “visible light” or “visible emission” and similar terms refer to light having one or more wavelengths in the range of about 380-780 nm. Herein, UV may especially refer to a wavelength selected from the range of 190-380 nm, such as 200-380 nm. The terms “violet light” or “violet emission”, and similar terms, may especially relate to light having a wavelength in the range of about 380-440 nm. In specific embodiments, the violet light may have a centroid wavelength in the 380-440 nm range. The terms “blue light” or “blue emission”, and similar terms, may especially relate to light having a wavelength in the range of about 440-490 nm (including some violet and cyan hues). In specific embodiments, the blue light may have a centroid wavelength in the 440-490 nm range. The terms “green light” or “green emission”, and similar terms, may especially relate to light having a wavelength in the range of about 490-560 nm. In specific embodiments, the green light may have a centroid wavelength in the 490-560 nm range. The terms “yellow light” or “yellow emission”, and similar terms, may especially relate to light having a wavelength in the range of about 560-590 nm. In specific embodiments, the yellow light may have a centroid wavelength in the 560-590 nm range. The terms “orange light” or “orange emission”, and similar terms, may especially relate to light having a wavelength in the range of about 590-620 nm. In specific embodiments, the orange light may have a centroid wavelength in the 590-620 nm range. The terms “red light” or “red emission”, and similar terms, may especially relate to light having a wavelength in the range of about 620-750 nm. In specific embodiments, the red light may have a centroid wavelength in the 620-750 nm range. The terms “cyan light” or “cyan emission”, and similar terms, especially relate to light having a wavelength in the range of about 490-520 nm. In specific embodiments, the cyan light may have a centroid wavelength in the 490-520 nm range. The terms “amber light” or “amber emission”, and similar terms, may especially relate to light having a wavelength in the range of about 585-605 nm, such as about 590-600 nm. In specific embodiments, the amber light may have a centroid wavelength in the 585-605 nm range. The phrase “light having one or more wavelengths in a wavelength range” and similar phrases may especially indicate that the indicated light (or radiation) has a spectral power distribution with at least intensity or intensities at these one or more wavelengths in the indicate wavelength range. For instance, a blue emitting solid state light source will have a spectral power distribution with intensities at one or more wavelengths in the 440-495 nm wavelength range.

The term “centroid wavelength”, also indicated as Δc, is known in the art, and refers to the wavelength value where half of the light energy is at shorter and half the energy is at longer wavelengths; the value is stated in nanometers (nm). It is the wavelength that divides the integral of a spectral power distribution into two equal parts as expressed by the formula λc=Σλ*I(λ)/(ΣI(λ)), where the summation is over the wavelength range of interest, and I(λ) is the spectral energy density (i.e. the integration of the product of the wavelength and the intensity over the emission band normalized to the integrated intensity). The centroid wavelength may e.g. be determined at operation conditions.

### BRIEF DESCRIPTION OF THE DRAWINGS

Embodiments of the invention will now be described, by way of example only, with reference to the accompanying schematic drawings in which corresponding reference symbols indicate corresponding parts, and in which:

FIGS. 1a-1c schematically show some spectral options;

FIGS. 2a-2c schematically show some embodiments; and

FIG. 3 schematically depict some application embodiments.

The schematic drawings are not necessarily to scale.

### DETAILED DESCRIPTION OF THE EMBODIMENTS

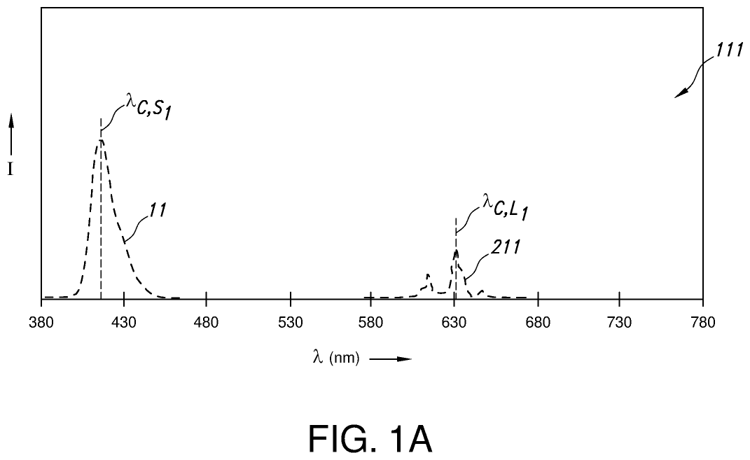

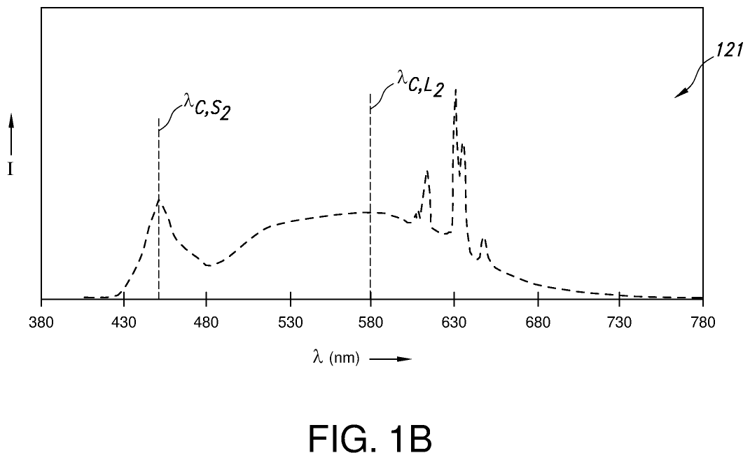

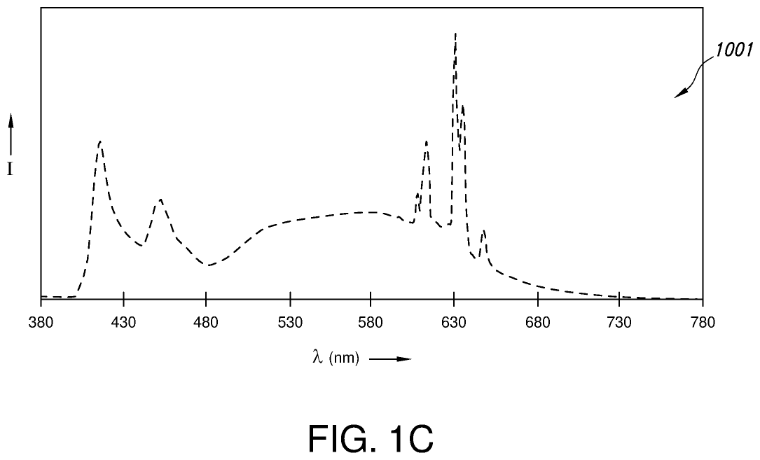

FIG. 1a schematically depicts a spectral power distribution of a (deep) blue solid state light source and a red emitting luminescent material. This may be an embodiment of first device light 111. Here, the luminescent material is a KSF type luminescent material. Reference 211 indicates first converter light and reference 11 indicates first light source light. Note that other spectral power distributions may also be possible. The respective centroid wavelengths are indicated, but see also below.

FIG. 1b schematically depicts a spectral power distribution of a blue solid state light source and a yellow-red emitting luminescent material. This may be an embodiment of second device light 121. Here, the luminescent material comprises a garnet type of luminescent material, an (oxy) nitride type luminescent material, and a KSF type luminescent material. Reference 221 indicates second converter light and reference 21 indicates first light source light. Note that other spectral power distributions may also be possible. The respective centroid wavelengths are indicated, but see also below. Note that the luminescent converter light includes contributions of luminescent material light of different luminescent materials.

FIG. 1c schematically depicts a spectral power distribution of system light 1001 comprising the first device light 111 and the second device light 121, like e.g. provided in FIGS. 1a-1b.

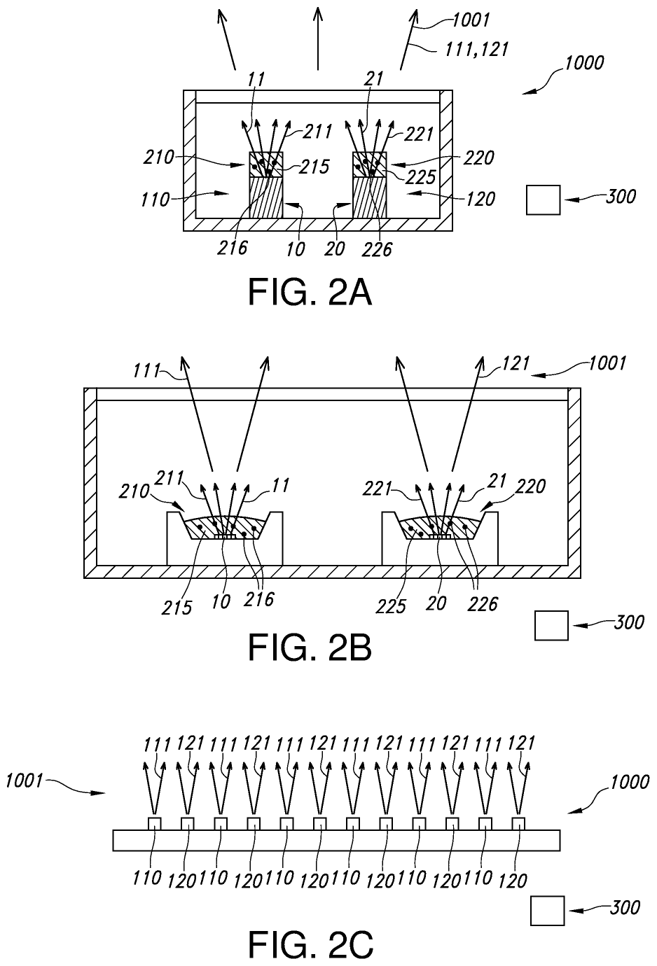

FIGS. 2a-2c schematically depict some embodiments of light generating systems 1000. Referring to these drawings the light generating system 1000 may be configured to generate system light 1001.

The light generating system 1000 may comprise a first light generating device 110 and a second light generating device 120. The first light generating device 110 may comprise a first light source 10 and a first luminescent converter 210. The first light source 10 may comprise a solid state light source. The first light source 10 may be configured to generate first light source light 11 having a first light source centroid wavelength (λ_{c,S1}) selected from the wavelength range of 380-420 nm. The first luminescent converter 210 may be configured to convert at least part of the first light source light 11 into first luminescent converter light 211 having a first luminescent converter centroid wavelength (λ_{c,L1}) selected from the wavelength range of 590-780 nm. The first luminescent converter 210 may comprise a first matrix material 215 and a first luminescent material 216. The first luminescent material 216 has a first weight percentage CW₁ relative to a total weight of the first luminescent converter 216. The first light generating device 110 may be configured to generate first device light 111 having a spectral power distribution in the wavelength range of 380-780 nm with at least 60% of a spectral power provided by the first light source light 11 and at maximum 40% of the spectral power provided by the first converter light 211.

The second light generating device 120 may comprise a second light source 20 and a second luminescent converter 220. The second light source 20 may comprise a solid state light source. The second light source 20 may be configured to generate second light source light 21 having a second light source centroid wavelength (λ_{c,S2}) selected from the wavelength range of 430-490 nm. The second luminescent converter 220 may be configured to convert at least part of the second light source light into second converter light 221 having a second luminescent converter centroid wavelength (λ_{c,L2}) selected from the wavelength range of 490-780 nm. The second luminescent converter 220 may comprise a second matrix material 225 and a second luminescent material 226. The second luminescent material 226 has a second weight percentage CW₂ relative to a total weight of the second luminescent converter 220. The second light generating device 120 may be configured to generate second device light 121 having a spectral power distribution in the wavelength range of 380-780 nm with at least 60% of the spectral power provided by the second converter light 221 and at maximum 40% of the spectral power provided by the second light source light 21.

Especially, in embodiments CW₁/CW₂≤0.5, such as 0.02≤CW₁/CW₂≤0.5.

The first light source 10 and the second light source 20 may be selected from the group of light emitting diodes, laser diodes and superluminescent diodes.

The light generating system 1000 may be configured to provide in an operational mode white system light 1001. The light generating devices 110,120 may be selected such to provide the system light 1001. The color rendering index may be at least 80 and the correlated color temperature may be selected from the range of 1800-6500 K.

The first device light 111 may have a spectral power distribution in the wavelength range of 380-780 nm with at maximum 85% of a spectral power provided by the first light source light 11 and at minimum 15% of the spectral power provided by the first converter light 211. The second device light 121 may have a spectral power distribution in the wavelength range of 380-780 nm with at maximum 85% of the spectral power provided by the second converter light 221 and at minimum 15% of the spectral power provided by the second light source light 21.

The first luminescent material 216 may comprise one or more of a Eu²⁺-based luminescent material and a Mn⁴⁺-based luminescent material. The second luminescent material 226 may comprise one or more of a Eu²⁺-based luminescent material and a Mn⁴⁺-based luminescent material. The first luminescent material 216 and the second luminescent material 226 comprise one or more luminescent materials individually selected from the group MS:Eu²⁺, M₂Si₅N₈:Eu²⁺, MAlSiN₃:Eu²⁺, Ca₂AlSi₃O₂N₅:Eu²⁺, and M′ₓM″₂₋₂ₓAX₆ doped with tetravalent manganese. Herein, M may comprise one or more of Ba, Sr, and Ca, especially in embodiments at least Sr. M′ may comprise an alkaline earth cation. M″ may comprise a cation, and x may be in the range of 0-1. Further, herein A may comprise a tetravalent cation. X may comprise a monovalent anion, at least comprising fluorine. The first luminescent material 216 and the second luminescent material 226 differ in composition.

In embodiments, (a) the first luminescent material 216 may comprise one or more of MS:Eu²⁺, M₂Si₅N₈:Eu²⁺, MAlSiN₃:Eu²⁺, Ca₂AlSi₃O₂N₅:Eu²⁺, and (b) the second luminescent material 226 may comprise one or more of M′ₓM″₂₋₂ₓAX₆ doped with tetravalent manganese. In embodiments, (a) the second luminescent material 226 does not may comprise one or more of MS:Eu²⁺, M₂Si₅N₈:Eu²⁺, MAlSiN₃:Eu²⁺, Ca₂AlSi₃O₂N₅:Eu²⁺, and (b) the first luminescent material 216 does not may comprise one or more of M′ₓM″₂₋₂ₓAX₆ doped with tetravalent manganese.

In specific embodiments, (a) the second luminescent material 226 may comprise one or more of MS:Eu²⁺, M₂Si₅N₈:Eu²⁺, MAlSiN₃:Eu²⁺, Ca₂AlSi₃O₂N₅:Eu²⁺, and (b) the first luminescent material 216 may comprise M′ₓM″₂₋₂ₓAX₆ doped with tetravalent manganese. In embodiments, (a) the first luminescent material 216 does not may comprise one or more of MS:Eu²⁺, M₂Si₅N₈:Eu²⁺, MAlSiN₃:Eu²⁺, Ca₂AlSi₃O₂N₅:Eu²⁺, and (b) the second luminescent material 226 does not may comprise one or more of M′ₓM″₂₋₂ₓAX₆ doped with tetravalent manganese.

In specific embodiments, the second light generating device 120 may be configured to generate white second device light 121. The color rendering index of the second device light 121 may be lower than the color rendering index of the system light 1001 as defined above. In specific embodiments, the second light generating device 120 may be configured to generate second device light 121 having a color point that may be configured further away from the black body locus than the color point of the white system light 1001 (comprising the first device light 111 and the second device light 121). In specific embodiments, the second light generating device 120 may be configured to generate second device light 121 having a color point of at least 15 Standard Deviation of Color Matching from the black body locus.

In specific embodiments, the first light source centroid wavelength (λ_{c,S1}) may be selected from the range of 400-420 nm, and/or the second light source centroid wavelength (λ_{c,S2}) may be selected from the range of 430-470 nm, more especially 440-470 nm.

In specific embodiments, the first matrix material 215 and the second matrix material 225 may individually be selected from an organic polymeric material, a silicone, and a (non-luminescent) silicate, especially may comprise cross-linked poly dimethyl siloxane (cross-linked PDMS).

FIG. 2a schematically depicts solid state light sources 10,20 with luminescent converter material 210,220 on the respective (dies of the) solid state light sources 10,20, such as LEDs. The respective light generating devices 110,120 are configured in a housing comprising a light transmissive window, e.g. from polymeric material or glass. Here, the housing is schematically depicted as having a rectangular cross-section. Other types of shapes may also be possible. FIG. 2b is a variant thereon, wherein the luminescent converter material 210,220 and solid state light sources 10,20 are configured in light reflective cups. In a non-depicted embodiments the first light generating device 110 and the second light generating device 120 may be configured in a single light reflective cup. In a variant on FIGS. 2a-2b, both the light generating devices 110,120 may also be configured in a single (larger) light reflective cup, with or without a light transmissive window. FIG. 2c schematically depicts an embodiment of a LED strip (or LED filament). Other embodiments than schematically depicted in FIGS. 2a-2c may also be possible.

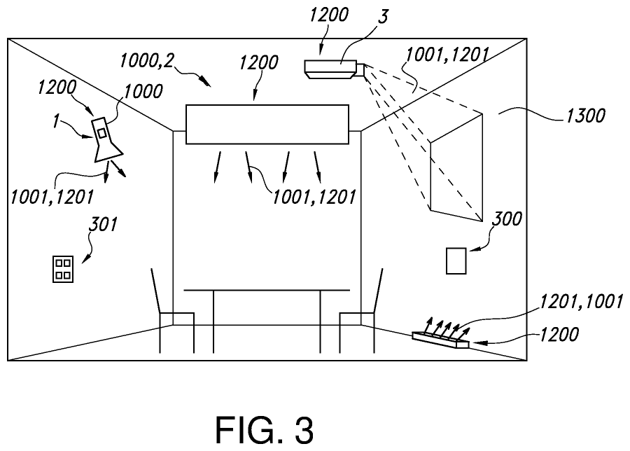

FIG. 3 schematically depicts an embodiment of a luminaire 2 comprising the light generating system 1000 as described above. Reference 301 indicates a user interface which may be functionally coupled with the control system 300 comprised by or functionally coupled to the light generating system 1000. FIG. 3 also schematically depicts an embodiment of lamp 1 comprising the light generating system 1000. Reference 3 indicates a projector device or projector system, which may be used to project images, such as at a wall, which may also comprise the light generating system 1000. Hence, FIG. 3 schematically depicts embodiments of a lighting device 1200 selected from the group of a lamp 1, a luminaire 2, a projector device 3, a disinfection device, a photochemical reactor, and an optical wireless communication device, comprising the light generating system 1000 as described herein. In embodiments, such lighting device may be a lamp 1, a luminaire 2, a projector device 3, a disinfection device, or an optical wireless communication device. Lighting device light escaping from the lighting device 1200 is indicated with reference 1201. Lighting device light 1201 may essentially consist of system light 1001, and may in specific embodiments thus be system light 1001. Reference 1300 refers to a space, such as a room.

In embodiments, a method for treating at least part of a space 1300 or of an object, may be applied, wherein especially the method may comprise providing system light 1001 comprising the first device light in the space 1300 or to the object, using the light generating system 1000 as defined herein or the lighting device 1200 as defined herein. Especially, the system light may also comprise second device light.

The term “plurality” refers to two or more. The terms “substantially” or “essentially” herein, and similar terms, will be understood by the person skilled in the art. The terms “substantially” or “essentially” may also include embodiments with “entirely”, “completely”, “all”, etc. Hence, in embodiments the adjective substantially or essentially may also be removed. Where applicable, the term “substantially” or the term “essentially” may also relate to 90% or higher, such as 95% or higher, especially 99% or higher, even more especially 99.5% or higher, including 100%. The term “comprise” also includes embodiments wherein the term “comprises” means “consists of”. The term “and/or” especially relates to one or more of the items mentioned before and after “and/or”. For instance, a phrase “item 1 and/or item 2” and similar phrases may relate to one or more of item 1 and item 2. The term “comprising” may in an embodiment refer to “consisting of” but may in another embodiment also refer to “containing at least the defined species and optionally one or more other species”. Use of the verb “to comprise” and its conjugations does not exclude the presence of elements or steps other than those stated in a claim. Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”. The article “a” or “an” preceding an element does not exclude the presence of a plurality of such elements.

Furthermore, the terms first, second, third and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. Note that “first” in the term “first luminescent material” is thus only an indication, but will in general not refer to a second luminescent material (and vice versa) (in view of the appropriate circumstances).

The devices, apparatus, or systems may herein amongst others be described during operation. As will be clear to the person skilled in the art, the invention is not limited to methods of operation, or devices, apparatus, or systems in operation.

It should be noted that the above-mentioned embodiments illustrate rather than limit the invention, and that those skilled in the art will be able to design many alternative embodiments without departing from the scope of the appended claims.

In the claims, any reference signs placed between parentheses shall not be construed as limiting the claim.

The invention may be implemented by means of hardware comprising several distinct elements, and by means of a suitably programmed computer. In a device claim, or an apparatus claim, or a system claim, enumerating several means, several of these means may be embodied by one and the same item of hardware. In yet a further aspect, the invention (thus) provides a software product, which, when running on a computer is capable of bringing about (one or more embodiments of) the method as described herein.

The invention also provides a control system that may control the device, apparatus, or system, or that may execute the herein described method or process. Yet further, the invention also provides a computer program product, when running on a computer which is functionally coupled to or comprised by the device, apparatus, or system, controls one or more controllable elements of such device, apparatus, or system.

1. A light generating system configured to generate system light, wherein the light generating system comprises a first light generating device and a second light generating device; wherein:
the first light generating device comprises a first light source and a first luminescent converter; wherein the first light source comprises a solid state light source, wherein the first light source is configured to generate first light source light having a first light source centroid wavelength selected from the wavelength range of 380-420 nm; wherein the first luminescent converter is configured to convert at least part of the first light source light into first luminescent converter light having a first luminescent converter centroid wavelength selected from the wavelength range of 590-780 nm; wherein the first luminescent converter comprises a first matrix material and a first luminescent material, wherein the first luminescent material has a first weight percentage CW₁ relative to a total weight of the first luminescent converter;
the first light generating device is configured to generate first device light having a spectral power distribution in the wavelength range of 380-780 nm with at least 60% of a spectral power provided by the first light source light and at maximum 40% of the spectral power provided by the first converter light;
the second light generating device comprises a second light source and a second luminescent converter; wherein the second light source comprises a solid state light source, wherein the second light source is configured to generate second light source light having a second light source centroid wavelength selected from the wavelength range of 430-490 nm; wherein the second luminescent converter is configured to convert at least part of the second light source light into second converter light having a second luminescent converter centroid wavelength selected from the wavelength range of 490-780 nm; wherein the second luminescent converter comprises a second matrix material and a second luminescent material, wherein the second luminescent material has a second weight percentage CW₂ relative to a total weight of the second luminescent converter;
the second light generating device is configured to generate second device light having a spectral power distribution in the wavelength range of 380-780 nm with at least 60% of the spectral power provided by the second converter light and at maximum 40% of the spectral power provided by the second light source light; and
CW1/CW2≤0.5.

2. The light generating system according to claim 1, wherein:
the first light source and the second light source are selected from the group of light emitting diodes, laser diodes and superluminescent diodes;
the light generating system is configured to provide in an operational mode white system light comprising the first device light and the second device light; wherein the light generating devices are selected such to provide the system light wherein the color rendering index is at least 80 and the correlated color temperature is selected from the range of 1800-6500 K;
the first device light has a spectral power distribution in the wavelength range of 380-780 nm with at maximum 85% of a spectral power provided by the first light source light and at minimum 15% of the spectral power provided by the first converter light; and
the second device light has a spectral power distribution in the wavelength range of 380-780 nm with at maximum 85% of the spectral power provided by the second converter light and at minimum 15% of the spectral power provided by the second light source light.

3. The light generating system according to claim 1, wherein the first luminescent material comprises one or more of a Eu2+-based luminescent material and a Mn4+-based luminescent material, and wherein the second luminescent material comprises one or more of a Eu2+-based luminescent material and a Mn4+-based luminescent material.

4. The light generating system according to claim 1, wherein the first luminescent material and the second luminescent material comprise one or more luminescent materials individually selected from the group MS:Eu2+, M2Si5N8:Eu2+, MAlSiN3:Eu2+, Ca2AlSi3O2N5:Eu2+, and M′xM″2−2xAX6 doped with tetravalent manganese, wherein M comprises one or more of Ba, Sr, and Ca, especially in embodiments at least Sr; wherein M′ comprises an alkaline earth cation, wherein M″ comprises a cation, and x is in the range of 0-1, wherein A comprises a tetravalent cation, wherein X comprises a monovalent anion, at least comprising fluorine; and wherein the first luminescent material and the second luminescent material differ in composition.

5. The light generating system according to claim 4, wherein (a) the first luminescent material comprises one or more of MS:Eu2+, M2Si5N8:Eu2+, MAlSiN3:Eu2+, Ca2AlSi3O2N5:Eu2+, and (b) the second luminescent material comprises one or more of M′xM″2−2xAX6 doped with tetravalent manganese.

6. The light generating system according to claim 5, wherein (a) the second luminescent material does not comprise one or more of MS:Eu2+, M2Si5N8:Eu2+, MAlSiN3:Eu2+, Ca2AlSi3O2N5:Eu2+, and (b) the first luminescent material does not comprise one or more of M′xM″2−2xAX6 doped with tetravalent manganese.

7. The light generating system according to claim 4, wherein (a) the second luminescent material comprises one or more of MS:Eu2+, M2Si5N8:Eu2+, MAlSiN3:Eu2+, Ca2AlSi3O2N5:Eu2+, and (b) the first luminescent material comprises M′xM″2-2xAX6 doped with tetravalent manganese.

8. The light generating system according to claim 7, wherein (a) the first luminescent material does not comprise one or more of MS:Eu2+, M2Si5N8:Eu2+, MAlSiN3:Eu2+, Ca2AlSi3O2N5:Eu2+, and (b) the second luminescent material does not comprise one or more of M′xM″2−2xAX6 doped with tetravalent manganese.

9. The light generating system according to claim 1, wherein the second light generating device is configured to generate white second device light; and wherein the second luminescent converter comprises one or more second luminescent materials of the type A3B5O12:Ce, wherein A in embodiments comprises one or more of Y, La, Gd, Tb and Lu, especially (at least) one or more of Y, Gd, Tb and Lu, and wherein B in embodiments comprises one or more of Al, Ga, In and Sc.

10. The light generating system according to claim 1, wherein the second light generating device is configured to generate second device light having a color point that is configured further away from the black body locus than the color point of the system light as defined in claim 2.

11. The light generating system according to claim 1, wherein the first light source centroid wavelength is selected from the range of 400-420 nm, and wherein the second light source centroid wavelength is selected from the range of 440-470 nm.

12. The light generating system according to claim 1, wherein 0.02≤CW1/CW2≤0.5.

13. The light generating system according to claim 1, wherein the first matrix material and the second matrix material comprise cross-linked poly dimethyl siloxane.

14. A lighting device selected from the group of a lamp, a luminaire, a projector device, a disinfection device, a photochemical reactor, an automotive lighting device, and an optical wireless communication device, comprising the light generating system according to claim 1.

15. A method for treating at least part of a space or of an object, wherein the method comprises providing system light comprising first device light and second device light in the space or to the object, using the light generating system as defined in claim 1.
